(19) United States
(12) Patent Application Publication
(10) Pub. No.: US 2018/0128824 A1
(43) Pub. Date: May 10, 2018

(54) NOVEL DESIGN OF ENZYME-LINKED IMMUNOSORBENT ASSAY PLATES AND SYSTEMS AND METHODS OF USE THEREOF

(71) Applicant: AGENCY FOR SCIENCE, TECHNOLOGY AND RESEARCH, Singapore (SG)

(72) Inventors: Vigneshwaran MANI, Singapore (SG); Amit SINGHAL, Singapore (SG); Gennaro DE LIBERO, Singapore (SG)

(21) Appl. No.: 15/570,719

(22) PCT Filed: May 3, 2016
(86) PCT No.: PCT/SG2016/050204

§ 371 Date: Oct. 30, 2017

(30) Foreign Application Priority Data
Apr. 30, 2015 (SG) 10201503435Q

(51) Int. Cl. G01N 33/543; B01L 3/00; G01N 33/569; G01N 33/68

(52) U.S. Cl. CPC G01N 33/54386; B01L 2300/161; G01N 33/5695; G01N 33/6866; G01N 33/6863; G01N 33/6869; G01N 2333/35; G01N 2333/57; G01N 2333/525; G01N 2333/55; G01N 2800/60; B01L 2300/0877; B01L 2300/087; B01L 2300/08; B01L 3/5023

(57) ABSTRACT

The present disclosure refers to an enzyme-linked immunosorbent assay (ELISA) plate comprising at least one row of reaction chambers, wherein the reaction chambers in the same row are in fluid communication with each other. Also enclosed is a system for detecting one or more target analytes comprising an ELISA plate as described herein, a plurality of magnetic beads and a magnet configured to cooperate with the magnetic beads. Also encompassed is a method of performing an ELISA assay which comprises of moving magnetic beads through subsequent reaction chambers, wherein the reaction chambers are alternatingly filled with a non-aqueous liquid, such as silicone oil, and aqueous ELISA reagents.

### CROSS-REFERENCE TO RELATED APPLICATIONS

This application claims the benefit of priority of Singapore provisional application No. 10201503435Q, filed 30 Apr. 2015, the contents of it being hereby incorporated by reference in its entirety for all purposes.

### FIELD OF THE INVENTION

The present invention relates generally to the field of molecular biology. In particular, the present invention relates to the use of specially designed plates in the detection of analytes.

### BACKGROUND OF THE INVENTION

Management and control of many diseases, such as tuberculosis (TB) still remains a significant threat to public health, partly due to the absence of cost-effective, sensitive, and rapid diagnostic tests. For example, for tuberculosis, currently, sputum smear microscopy is the most commonly used point-of-care (POC) method for diagnosis in endemic countries, despite its poor sensitivity (30-60%). Although “gold standard” bacterial culture does provide the required sensitivity (>90%), the test takes several weeks and requires well-equipped laboratories and trained staff. Such a long turn-around time often results in delayed diagnosis, continued transmission, and the risk of developing drug resistance.

Serological tests based on the detection of antibodies against, for example, mycobacterial protein antigens for diseases like tuberculosis in the form of lateral flow devices or standard ELISAs have been extensively used for diagnostic purposes. However, like many ELISA tests, these tests have demonstrated poor sensitivity (1-60%) and specificity (53-99%) compared with standard culture methods, performing no better than sputum smear microscopy, and have failed to improve patient outcomes. As such, the World Health Organization (WHO) has recommended against the usage of serological tests. Endorsements by the WHO of nucleic acid amplification-based tuberculosis diagnostic tests, such as those known in the art and commercially available, have helped to fill this gap. However, their implementation in resource-limited settings has been severely restricted by high maintenance costs and the need for sophisticated instrumentation, trained personnel, and uninterrupted electrical supply. Thus, there is a need for a simple, sensitive, and portable assay for the early stage detection of tuberculosis at the point-of-care (POC).

### SUMMARY

In one aspect, the present invention refers to an enzyme-linked immunosorbent assay (ELISA) plate comprising at least one row of reaction chambers, wherein the reaction chambers in the same row are in fluid communication with each other.

In another aspect, the present invention refers to a system for detecting a target analyte comprising an enzyme-linked immunosorbent assay (ELISA) plate as disclosed herein, a plurality of magnetic beads and a magnet configured to cooperate with the magnetic beads.

In yet another aspect, the present invention refers to a kit comprising an enzyme-linked immunosorbent assay (ELISA) plate as disclosed herein, a plurality of magnetic beads and a magnet.

In a further aspect, the present invention refers to a method of performing an enzyme-linked immunosorbent assay (ELISA) using a system as disclosed herein, wherein the reaction chambers of the ELISA plate are liquid-filled, the method comprising (a) incubating a sample comprising one or more target analytes with a plurality of magnetic beads capable of capturing said one or more target analytes in the first chamber of each row of the ELISA plate according to any of the preceding claims; (b) loading the subsequent reaction chambers of the columns of the ELISA plate with alternating liquids, wherein the liquids are either aqueous or non-aqueous; (c) moving the plurality of magnetic beads from the first reaction chambers of each row to subsequent reaction chambers of the same row by using the magnet; (d) incubating the plurality of magnetic beads in subsequent reaction chambers; (e) repeating steps (c) to (d) until the final chamber in the row is reached, and (f) detecting the signal generated in the final reaction chamber.

In another aspect, the present invention refers to a method of detecting tuberculosis in a subject using the system as disclosed herein or the kit as disclosed herein.

In yet another aspect, the present invention refers to a method of detecting at least one cytokine in a sample using the system as disclosed herein or the kit as disclosed herein.

### BRIEF DESCRIPTION OF THE DRAWINGS

The invention will be better understood with reference to the detailed description when considered in conjunction with the non-limiting examples and the accompanying drawings, in which:

FIG. 1 shows the templates used for the generation of the ELISA plate as described herein. FIG. 1A shows an AutoCAD design template for microchip fabrication of the top part of microchip device, which was laser cut on 3 mm thick poly methyl methacrylate (PMMA). The numbers in the scheme refer to object dimension in millimetre. FIG. 1B shows an AutoCAD design template for microchip fabrication of the bottom part of microchip device, which as laser cut on 1.5 mm thick poly methyl methacrylate (PMMA). The numbers in the scheme refer to object dimension in millimetre. FIG. 1C shows the assembly of, for example, a hydrophobic film-bonded microchip device, wherein the bottom part of the microchip device (bottom layer) is bonded to the hydrophobic layer (for example parafilm; middle layer) and the top part of the microchip device (top layer).

FIG. 2 shows a general schematic of the ELISA plate as disclosed in the present description, both in use and when not in use. The schematic shows the ELISA plate when not in use and denotes a possible direction of movement of the magnetic beads.

FIG. 3 shows schematic representation of a microchip employing magnet-actuated magnetic bead ELISA for the simultaneous detection of glycolipid, protein, and a mixture of glycolipid and protein-specific IgG antibodies in the plasma of active tuberculosis (ATB), latent TB infection (LTBI) and healthy control (HC) individuals. The microchip consists of channel featuring interconnected chambers for aqueous reagent storage (circular) and non-aqueous silicone oil (rhombus). Microchip has six channels, with each channel featuring independent lipid or protein coated MBs and mixtures of lipid and protein MBs. For example, channel 1 contains trehalose 6,6′-dimycolate (TDM) coated magnetic beads, channel 2 contains 38 KDa coated magnetic beads, channel 3 contains Antigen 85A coated magnetic beads, channel 4 contains trehalose 6,6′-dimycolate (TDM) magnetic beads and 38 KDa magnetic beads, channel 5 contains trehalose 6,6′-dimycolate (TDM) magnetic beads and Antigen 85A magnetic beads, and channel 6 contains BSA coated magnetic beads (as a negative control). The magnetic beads in each chamber are simultaneously actuated using six magnets positioned underneath the chip from one reagent chamber to other through the silicone oil phase. Total time of the immunoassay is approximately 15 min from addition of plasma sample to detection of human antibody.

FIG. 4 shows photographs of magnet-actuated magnetic bead ELISA before (left) and after 15 minutes (right) from addition of plasma sample. Row A shows the magnetic beads for detecting trehalose 6,6′-dimycolate (TDM), row B shows the magnetic beads for detecting 38 kDa, row C shows the magnetic beads for detecting Ag85A, row D shows the magnetic beads for detecting trehalose 6,6′-dimycolate (TDM) and 38 kDA, row E shows the magnetic beads for detecting trehalose 6,6′-dimycolate (TDM) and Ag85A, row F is BSA (negative control). The odd numbered columns are circular reagent storage chambers and the even numbered columns are rhombic silicone oil chambers. Column 1 contains plasma samples and the respective coated magnetic beads, column 5 contains biotin conjugated anti-IgG antibody, column 9 contains streptavidin poly-horse radish peroxidase (HRP) antibody and column 13 contains 3′,3′,5′,5′-Tetramethylbezidine (TMB) substrate for colorimetric detection. Columns 3, 7, and 11 contain a wash buffer. Columns 2, 4, 6, 8, and 10 contain silicone oil.

FIG. 5 shows a representative schematic of hydrophobic film-bonded microchip ELISA for detection of IFN-γ and TNF-α in, for example phosphate buffered saline with Tween (PBS-T). MP stands for magnetic particles (just as MB stands for magnetic beads; MB); TMB stands for 3′,3′,5′,5′-Tetramethylbezidine, a substrate for colorimetric detection.

FIG. 6 shows data pertaining to the comparison of an ELISA performed using TDM coated magnetic beads in a conventional 96-well ELISA plate (here after know as ‘MB ELISA’) and a conventional 96-well plate ELISA (here after know as ‘conventional plate ELISA’). In all the ELISA-based experiments, TDM beads had a TDM concentration of about 0.41 μg/cm² on their surface (pleas also see FIG. 12 below). FIG. 6A shows a scatter plot illustrating the comparison of the distribution of anti-trehalose 6,6′-dimycolate (anti-TDM) IgG responses in the plasma of active TB (ATB) and healthy control (HC) individuals determined using MB ELISA, where plasma was diluted 125-fold in 5% BSA buffer. N=40, ATB=19 and HC=21 (***, P=0.0003). FIG. 6B shows a scatter plot illustrating the comparison of the distribution of anti-trehalose 6,6′-dimycolate (anti-TDM) IgG responses in the plasma of active tuberculosis (ATB) and healthy control (HC) individuals determined using conventional plate ELISA where plasma was diluted 2500-fold in 5% BSA. N=40, ATB=19 and HC=21 (***, P=0.0007 for plate ELISA). FIG. 6C shows a line graph illustrating the linear regression between the magnetic bead ELISA (MB ELISA) and the conventional plate ELISA, thereby showing the correlation of anti-TDM plasma IgG responses using the two different ELISA assays.

FIG. 7 shows the line graph of the standard curve of IFN-γ, TNF-α, and IL-2 spiked in RPMI medium+10% FCS measured using parafilm coated 4.5 m bead microchip ELISA assay for test concentrations ranging between 10 pg/ml to 2500 pg/ml. DL represents detection limit, which was calculated based on test signal>average blank (0 pg/ml)+3 stdev. The curves were fitted using nonlinear regression-second order polynomial (quadratic) using Graphpad Prism.n=3. The detection limit for IFN-γ is 20 pg/ml (FIG. 7A). The detection limit for TNF-α is 40 pg/ml (FIG. 7B). The detection limit for IL-2 is 40 pg/ml (FIG. 7C).

FIG. 8 shows data derived from the analysis of the results of the magnetic bead based microchip ELISA comparing active tuberculosis (ATB), latent tuberculosis infection (latent TB or LTB) and healthy individuals (H or HC). FIG. 8A shows a scatter plot illustrating the distribution of antibody response against Mycobacterium tuberculosis trehalose 6,6′-dimycolate (TDM) in the plasma of ATB, LTB and H individuals, which was determined by microchip immunoassay. The scatter plots represent comparison of Mycobacterium tuberculosis antigen specific antibody distribution between different groups, i.e. ATB vs. LTB (left), ATB vs. H (centre) and LTB vs. H (right). The dots in the scatter plot represent plasma from a single individual. Horizontal lines are cut-off values determined to measure sensitivity at fixed specificity of 75%. Each scatter plot consists of the values of sensitivity and specificity based on the comparison of different groups. The total number of plasma samples=146, ATB=65, LTB=40, and H=41. FIG. 8B shows a scatter plot illustrating the distribution of antibody response against 38 kDa in the plasma of ATB, LTB and H individuals, which was determined by microchip immunoassay. The scatter plots represent comparison of Mycobacterium tuberculosis antigen specific antibody distribution between different groups, i.e. ATB vs. LTB (left), ATB vs. H (centre) and LTB vs. H (right). The dots in the scatter plot represent plasma from a single individual. Horizontal lines are cut-off values determined to measure sensitivity at fixed specificity of 75%. Each scatter plot consists of the values of sensitivity and specificity based on the comparison of different groups. The total number of plasma samples=146, ATB=65, LTB=40, and H=41. FIG. 8C shows a scatter plot illustrating the distribution of antibody response against Antigen 85A in the plasma of ATB, LTB and H individuals, which was determined by microchip immunoassay. The scatter plots represent comparison of Mycobacterium tuberculosis antigen specific antibody distribution between different groups, i.e. ATB vs. LTB (left), ATB vs. H (centre) and LTB vs. H (right). The dots in the scatter plot represent plasma from a single individual. Horizontal lines are cut-off values determined to measure sensitivity at fixed specificity of 75%. Each scatter plot consists of the values of sensitivity and specificity based on the comparison of different groups. The total number of plasma samples=146, ATB=65, LTB=40, and H=41. FIG. 8D shows a scatter plot illustrating the distribution of antibody response against mixture of 38 kDa with trehalose 6,6′-dimycolate (TDM) in the plasma of ATB, LTB and H individuals, which was determined by microchip immunoassay. The scatter plots represent comparison of Mycobacterium tuberculosis antigen specific antibody distribution between different groups, i.e. ATB vs. LTB (left), ATB vs. H (centre) and LTB vs. H (right). The dots in the scatter plot represent plasma from a single individual. Horizontal lines are cut-off values determined to measure sensitivity at fixed specificity of 75%. Each scatter plot consists of the values of sensitivity and specificity based on the comparison of different groups. The total number of plasma samples=146, ATB=65, LTB=40, and H=41. FIG. 8E shows a scatter plot illustrating the distribution of antibody response against mixture of antigen 85A with trehalose 6,6′-dimycolate (TDM) in the plasma of ATB, LTB and H individuals, which was determined by microchip immunoassay. The scatter plots represent comparison of Mycobacterium tuberculosis antigen-specific antibody distribution between different groups, i.e. ATB vs. LTB (left), ATB vs. H (centre) and LTB vs. H (right). The dots in the scatter plot represent plasma from a single individual. Horizontal lines are cut-off values determined to measure sensitivity at fixed specificity of 75%. Each scatter plot consists of the values of sensitivity and specificity based on the comparison of different groups. The total number of plasma samples=146, ATB=65, LTB=40, and H=41.

FIG. 9 shows graphs depicting the Receiver Operating Characteristic (ROC) curves for plasma IgG assays for individual antigens or combinations determined using microchip immunoassay for the scenarios: active tuberculosis versus latent tuberculosis, active tuberculosis versus healthy individual and latent tuberculosis infection versus healthy individual. FIG. 9A shows a line graph showing ROC curves for plasma IgG assays for trehalose 6,6′-dimycolate (TDM), 38 kDa, Antigen 85A, trehalose 6,6′-dimycolate (TDM) together with 38 kDa, and trehalose 6,6′-dimycolate (TDM) together with Antigen 85A for differentiating Active (ATB) from Latent (LTBI) individuals. The Area under the Curve (AUC) was calculated using the Graph Pad prism 5 software. FIG. 9B shows a line graph showing ROC curves for plasma IgG assays for trehalose 6,6′-dimycolate (TDM), 38 kDa, Antigen 85A, trehalose 6,6′-dimycolate (TDM) and 38 kDa, and trehalose 6,6′-dimycolate (TDM) and Antigen 85A for differentiating Active (ATB) from Healthy (HC) individuals. The AUC was calculated using the Graph Pad prism 5 software. FIG. 9C shows a line graph showing ROC curves for plasma IgG assays for trehalose 6,6′-dimycolate (TDM), 38 kDa, Antigen 85A, trehalose 6,6′-dimycolate (TDM) and 38 kDa, and trehalose 6,6′-dimycolate (TDM) and Antigen 85A for differentiating Latent (LTBI) from Healthy (HC) individuals. The AUC was calculated using the Graph Pad prism 5 software.

FIG. 10 shows a heat map depicting the reactivity of plasma to individual antigens, and their combinations as assessed by the microchip ELISA. Each column represents the response observed in one plasma sample and each row depicts the response to different antigens or their combinations. Normalised optical densities (OD) values are visualised as a colour spectrum as shown in the row z-scores. The heat map was generated using R statistical computing software, using z-score=(x−μ)/σ, where x is an individual's OD response, μ is mean of OD response from all individuals (N=146) for each antigen and a is the standard deviation. N=146; ATB=65; LTBI=40; HC=41.

FIG. 11 show scatterplots representing the data from the comparison of the results obtained from a sputum smear and cell culture, which are the current standard of care tests that are performed for identifying and characterising a tuberculosis infection. FIG. 11A shows a scatter plot illustrating the distribution of anti-trehalose 6,6′-dimycolate (anti-TDM) IgG response among classified active tuberculosis (ATB) samples. Samples were classified based on AFB (Acid fast bacilli) sputum smear grade. It is noted that the AFB sputum smear grade is applied in the clinic to estimate the bacillary load in the sputum of a patient. The higher the bacillary load, the more positive the grade. However, the resulting therapy is not dependent on the grade gained through this analysis. N=62; −ve=28; 1+=11; 2+=11; 3+=8 and 4+=4 (**, P=0.002 *, P=0.013). FIG. 11B shows a scatter plot illustrating the distribution of anti-trehalose 6,6′-dimycolate (anti-TDM) IgG response among classified active tuberculosis samples. Samples were classified according to a mycobacterial culture test (considered to be the current gold standard for tuberculosis diagnosis). Culture positive, N=47; culture negative N=13 (*P=0.0312).

FIG. 12 shows column graphs depicting the dynamic light scattering (DLS) of TDM-coated MB preparations with varying nominal TDM surface concentrations. A) 0 μg/cm², B) 0.16 μg/cm², C) 0.41 μg/cm², and D) 0.65 μg/cm². The mean diameter of each bead preparation was estimated using the Brookhaven particle size analyzer software.

FIG. 13 shows images of light microscope depicting uniform monodispersed, coated, magnetic beads. A) BSA-coated MBs and B) TDM-coated MBs (0.41 μg/cm²), at 400× magnification.

FIG. 14 shows the results of a thin layer chromatography analysis (TLC) of TDM extracted from magnetic beads. TDM standards and extracted TDM from different magnetic bead preparations of a small batch (0.2 ml) (i.e. with varying nominal TDM surface concentration, 0-0.65 μg/cm²) were spotted and TLC was carried out using CHCl₃/CH₃OH/H₂O (65:24:4) as mobile phase and orcinol-based carbohydrate staining. A linear calibration curve of TDM standard was used to quantify the TDM present on each bead preparation. The table below represents varying TDM loading onto 4×10⁷ beads (total surface area: 25.6 cm²), the recovered bound TDM, and the percentage (%) yield of bound TDM on each bead preparation.

FIG. 15 shows a graph depicting the flow cytometry based detection of anti-TDM IgG response in pooled active tuberculosis (ATB) plasma (N=5) using magnetic beads coated with varying nominal surface TDM concentrations (0-0.65 μg/cm²). Curves represent MFI peaks of TDM-coated MB preparations (0-0.65 μg/cm) upon capture of anti-TDM antibodies from pooled ATB plasma, which was then stained with Alexa-647 conjugated secondary antibodies. Samples were acquired using flow cytometer (MACS quant analyzer).

FIG. 16 shows a line graph depicting the data from the analysis of the stability of TDM-coated MBs (0.41 μg/cm²) at room temperature (22-25° C.) over a period of 10 months. Anti-IgG response in an active tuberculosis (ATB) patient plasma was measured using the microchip immunoassay as described herein at different days, and % relative activity at time (t, days) was obtained from initial activity of beads at day zero. N=3.

FIG. 17 shows a line graph depicting the data from a competition of TDM plate ELISA using free trehalose. Anti-TDM IgG levels were estimated using conventional TDM plate ELISA, where plasma was pre-incubated with/without free trehalose (10%) for 1 h prior to addition onto TDM-coated plates. N=22 (*, P=0.037).

FIG. 18 shows a scatterplot depicting the statistical distribution of anti-IgG response against 38 kDa and Ag85A in ATB patients. A and B) AFB smear graded sputum samples. N=62; −ve=28; 1+=11; 2+=11; 3+=8 and 4+=4), (38 kDa; *, P=0.032). C and D) Samples stratified based on culture test. N=60; Culture positive samples N=47; Culture negative samples, N.=13. NS, not significant.

### DEFINITIONS

As used herein, the term “ELISA” refers to an enzyme-linked immunosorbent assay, a common laboratory technique used to measure the concentration of one or more target analytes (usually antibodies, proteins or antigens and the like) in a solution. The basic ELISA, or enzyme immunoassay (EIA), distinguishes itself from other antibody-based assays due to the separation of specific and non-specific interactions that occur via serial binding to a solid surface, usually a polystyrene multi-well plate (for example a 96-well plate), and an ELISA can achieve quantitative results. The various steps of an ELISA result in a coloured end-product or an emitted signal which can be quantified using an appropriate detector, whereby the amount of the emitted signal or coloured end-product correlates to the amount of analyte present in the original sample.

As used herein, the term “geometry” refers to the surface shape of a given item, for example the shape of a reaction chamber. As used herein, this term can refer to the two-dimensional (2D) or three-dimensional (3D) shape. Thus, the use of the term square in reference to the geometry of the reaction chamber is referring to the two-dimensional shape of the reaction chamber. According to this line of thought, the use of the term cylindrical would therefore refer to the three dimensional shape of the reaction chamber.

As used herein, the term “reaction chambers” refers to the perforations within the top plate of the microchip ELISA plate as described herein, which together with the solid bottom plate form wells in which reaction liquids can be deposited and wherein the reactions of the ELISA take place.

As used herein, the term “hydrophobic” or “hydrophobicity” refers to the physical property of a molecule (then known as a hydrophobe) that is seemingly repelled from a mass of water. Strictly speaking, hydrophobicity does not involve a repulsive force, instead it is an absence of an attractive force. As known in the art, hydrophobic molecules tend to be non-polar (that is they do not have an overall dipole, or molecular dipole moment within the molecule) and, thus, prefer other neutral molecules and non-polar solvents. Hydrophobic molecules in water often cluster together, forming micelles or similar structures. Water on hydrophobic surfaces will exhibit a high contact angle. Examples of hydrophobic molecules include, but are not limited to, alkanes, oils, fats, and greasy substances in general. The term “hydrophobic” can be used interchangeably with the term “lipophilic” (that is “fat-loving”). However, it is to be noted that the two terms are not synonymous. While hydrophobic substances are known in the art to be lipophilic, there are exceptions of hydrophobic substances which are not lipophilic, such as the silicones and fluorocarbons.

As used herein, the term “aqueous” refers to an aqueous solution, which is a solution in which the solvent is water. It is usually shown in chemical equations by appending (aq) to the relevant chemical formula. For example, a solution of table salt, or sodium chloride (NaCl), in water would be represented as NaCl(aq). The word aqueous means pertaining to, related to, similar to, or dissolved in water. As water is an excellent solvent and is also naturally abundant, it is a ubiquitous solvent in chemistry. It is known in the art that substances that are termed “hydrophobic” (that is ‘water-fearing’) often do not dissolve well in water, whereas those that are termed “hydrophilic” (that is ‘water-loving’) do. An example of a hydrophilic substance is sodium chloride, as sodium chloride readily dissolves in water. The term used to describe the opposite effect, that is a solution wherein the solvent is not water, is termed “non-aqueous”.

As used herein, the concept of chemical polarity describes a separation of electric charge leading to a molecule or its chemical groups having an electric dipole or multi-pole moment. Polar molecules interact through dipole-dipole intermolecular forces and hydrogen bonds. Molecular polarity is dependent on the difference in electronegativity between atoms in a compound and the asymmetry of the compound's structure. Polarity underlies a number of physical properties including surface tension, solubility, and melting and boiling points. As used herein, the term “non-polar” refers to a molecule that is equally sharing the electrons between the two atoms of a diatomic molecule or a molecule that has a symmetrical arrangement of polar bonds, as is the case in more complex molecules. For example, boron trifluoride (BF₃) has a trigonal planar arrangement of three polar bonds at 120°. This results in no overall dipole in the molecule. An example of a polar molecule is hydrogen fluoride (HF), which is a linear molecule which has a dipole moment due to the high electronegativity of the fluoride atom, which results in the binding electrons as well as those of the hydrogen atom to be “pulled towards” the fluoride atom, resulting in partially negative charged region around the fluoride.

As used herein, the concept of miscibility refers to the properties of to mix in all proportions, forming a homogeneous solution. The term is most often applied to liquids, but applies also to solids and gases. Water and ethanol, for example, are miscible because they mix in all proportions. By contrast, the term “immiscible” is used to describe substances in which a significant proportion does not form a homogeneous solution. For example, butanone is considered to be significantly soluble in water, but these two solvents are not considered to be miscible (that is, they are immiscible) because they are not soluble in all proportions.

As used herein, the term “IgG” refers to a class of immunoglobulins, that include the most common antibodies circulating in the blood that facilitate the phagocytic destruction of microorganisms foreign to the body. These immunoglobulins bind to the microorganisms, or parts thereof, thereby activating the complement immune system. A naturally occurring antibody (e.g., IgG) includes four polypeptide chains, two heavy (H) chains and two light (L) chains inter-connected by disulfide bonds. However, it has been shown that the antigen-binding function of an antibody can be performed by fragments of a naturally occurring antibody. Thus, these antigen-binding fragments are also intended to be designated by the term “antibody.” Examples of binding fragments encompassed within the term antibody include (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment, which consists of a VH domain; and (v) an F(ab′)2 fragment, a bivalent fragment comprising two Fab fragments linked by a disulfide bridge at the hinge region. Furthermore, although the two domains of the Fv fragment are coded for by separate genes, a synthetic linker can be made that enables them to be made as a single protein chain (known as single chain Fv (scFv)) by recombinant methods. Such single chain antibodies, as well as dsFv, a disulfide stabilized Fv, and dimeric Fvs (diabodies), which are generated by pairing different polypeptide chains, are also included.

As used herein, the term “point-of-care (POC)”, also known as “bedside testing” refers as medical diagnostic testing at or near the point of care—that is, at the time and place of patient care. This is in contrast with the historical procedure, in which testing was wholly or mostly confined to the medical laboratory. This entailed sending off specimens away from the point of care and then waiting hours or days to learn the results, during which time care must continue without the desired information.

As used herein, the term “TDM” refers to trehalose 6,6′-dimycolate, a cord factor known to play an important role in tuberculosis infections.

As used herein, the term “38 kDa” refers to a 38-kDa lipoprotein, which is known in the art to induce macrophage caspase-dependent apoptosis during tuberculosis infection.

As used herein, the term “Antigen 85A (Ag85A)” refers to a secreted protein that is found in the most abundance in tuberculosis culture fluid.

As used herein, the term “serodiagnosis” refers to the diagnosis of a disease or a condition in a subject based on the study of the blood sera or any other serous fluid obtained from the subject. This can also include, but is not limited to, blood plasma, blood serum, whole blood, lymphatic fluid, urine, pleural effusions and serous fluid.

As used herein, the term “row” and “column” refer to the horizontal and the vertical orientation, respectively, of a line of reaction chambers on, for example, an ELISA plate. In this example, the row refers to a length of reaction chambers along the length of the ELISA plate, and the column refers to a length of reaction chambers along the width of the ELISA plate, wherein, the terms width and length are as defined herein, that is the width is the measurement along an edge of the object which is shorter than the length of the same object.

### DETAILED DESCRIPTION OF THE PRESENT INVENTION

The present disclosure provides a novel plate design for performing enzyme-linked immunosorbent assays, which can be used in miniaturised form for the simple, ultrafast, detection of, for example, IgG responses against multiple antigens or detection of cytokines, for example IFN-γ, in supernatant. This concept of detecting the presence of an immune molecule in a subject's body fluid is then used for the diagnosis and identification of a particular disease, for example, tuberculosis.

In one example, despite the identification of Mycobacterium tuberculosis as the cause of the disease tuberculosis more than a century ago, the diagnosis of tuberculosis in resource-limited settings continues to be a major challenge. Indeed, the same can be said, for example, for bacterial infections caused by similar bacterium or other infectious agents, which result in diseases or conditions with indefinite symptoms. For the vast majority of patients in endemic countries, for example, tuberculosis diagnosis, for example, depends primarily on sputum microscopy and culture, which have a number of limitations in sensitivity, specificity, and turnaround time. Access to point-of-care (POC), rapid, inexpensive, sensitive, and instrument-free tests for the diagnosis of tuberculosis (TB) remains a major challenge. A developed test must meet minimum specifications outlined by the World Health Organisation (WHO), such as speed of assay (less than 3 hours), minimal sample preparation, maintenance-free instrumentation, low-cost (less than $10 per test), and environmentally acceptable waste disposability.

Advances in micro-scale and nanoscale technologies offer a feasible approach for the development of miniaturised point-of-care devices. Micro-scale technologies allow integration and automation of multistep assays, such as enzyme-linked immunosorbent assay (ELISA), thus enabling sample processing, target capture, and detection into a single integrated device, which must otherwise be performed by a well-trained operator in a laboratory setting. In particular, magnetic beads (MB) have been exploited extensively in microfluidic ELISA because of their uniform size, high surface-to-volume ratio, fast reaction kinetics, and ease of manipulation, providing better sensitivity at a faster speed compared to conventional flat surfaces. Furthermore, with the use of an external magnet, magnetic beads can be actuated/manipulated through a series of stationary reagents for bio-detection in automated assays. This provides a simple ‘sample-in and answer-out’ based system, which is highly desirable for diagnosis at the point-of-care.

Presented herein is a microchip enzyme-linked immunosorbent assay, capable of detecting IgG responses against one or multiple antigens or any other target analytes (one or more) from plasma samples of active infections, for example active tuberculosis (ATB), in patients in a rapid and miniaturised detection system. Each microchip enzyme-linked immunosorbent assay, or enzyme-linked immunosorbent assay (ELISA) plate, can comprise between 5 to 10, between 8 to 15, between 18 to 32 rows, about 1, about 2, about 3, about 4, about 5, or about 6 individual rows. A marked technical characteristic of the enzyme-linked immunosorbent assay plate is the fact that the reaction chambers are in fluid communication with each other. In a conventional enzyme-linked immunosorbent assays, the sample analytes are bound to the surface of the reaction chambers using methods known in the art. The washing and changing of the fluids required for performing the enzyme-linked immunosorbent assay is usually done by tipping the ELISA plate over and tapping out any access reaction fluid. This kind of “man-handling” of samples is known to result in variances in measurements due to inadequate buffer removal, which can result in a dilution of the resulting signal. Also, the application of new buffers and reaction fluids by pipetting said fluids into the reaction chambers involves the risk of washing out the sample analyte before detection due to inadequate or forceful pipetting techniques, thereby introducing experimenter-specific signal variability. Therefore, in one example, the reaction chambers are in fluid communication with each other. This fluid communication enables the magnetic sample carrier with the target analyte (i.e. the magnetic beads) to be moved between each well without the need for any shifting of the plate or washing out of the target analyte, thereby reducing the risk of sample dilution and signal variation. Thus, in one example, there is disclosed an enzyme-linked immunosorbent assay (ELISA) plate comprising at least one row of reaction chambers, wherein the reaction chambers in the same row are in fluid communication with each other. The described fluid communication is enabled via openings in either or both walls of adjacent reaction chambers, thereby enabling the movement of the, for example magnetic beads, between the reaction chambers. This opening may also be termed as a “link” between the neighbouring reaction chambers. It is required that the openings between the adjacent reaction chambers are large enough to enable the movement of, for example, the magnetic beads between the reaction chambers, but also small enough that the fluids in each reaction chamber stay within their reaction chamber, that is to prevent, for example the non-aqueous liquids from flowing into the aqueous liquids deposited in the adjacent reaction chambers. In other words, for example, if the opening is too big, this large opening would assist in the spreading and mixing of the aqueous liquids used therein. If the opening is too small, it would make the movement (or actuation) of the magnetic beads to and from the adjacent chamber difficult. Therefore, the size of the opening is critical for efficient assay operation when using the claimed microchip ELISA. As mentioned previously, for example, a circular chamber would be a technically advantageous choice for the reaction chambers containing aqueous liquids, as the round shape provides uniformity in shape and in mixing, with equal surface tension along the edges. Furthermore, an opening in, for example, a circular reaction chamber provides easier access for the magnetic beads to transfer to adjacent, for example, rhombic reaction chambers. Surface tension, as well as the form or the geometry of the reaction chambers plays an important role in the separation of the different liquids.

The reaction chambers in the microchip as disclosed herein are linked (that is in fluid communication with each other) via one or more openings in the sides of the reaction chambers. As provided above, the size of these openings in the sides/walls of the reaction chambers plays an important role in the function of the present invention. In absolute terms, the described opening in the reaction chamber wall can be between 0.5 to 2 mm wide, between 1 to 1.8 mm wide, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm wide. In one example, the opening in the reaction chamber wall is 1.7 mm. In relative terms, the opening in the reaction chamber wall can be between 4% to 10%, or between 5% to 9% or about 6%, about 7%, about 8%, about 9% of, for example, the circumference of a circular chamber on one side of the reaction chamber. The opening in the reaction chamber wall on the opposite side of the reaction chamber would need to be of the same size in order to perform the same function. Therefore, the opening on the opposite side of the reaction chamber wall would also be between 4% to 10%, between 5% to 9%, or about 6%, about 7%, about 8%, about 9% of, for example, the circumference of a circular chamber on one side of the reaction chamber. In one example, the opening on the opposite side of the reaction chamber wall is between 5% to 9%.

In terms of handling of the invention as disclosed herein, or in regards to the loading of the reaction chambers as disclosed herein, it is understood that standard laboratory equipment, for example, such as a 20 μl, 100 μl, or 200 μl pipette, is used for liquid handling. A person skilled in the art would be capable of deciding which laboratory equipment would be appropriate for the desired experiment. For example, a 100 μl pipette can be employed to add the aqueous reagent into the circular chamber, whereas a 200 μl pipette can be used to fill in the oil chamber. In a working example, in the first step, unless otherwise mentioned, the aqueous liquids are first added into the circular reaction chambers on the microchip, after which the other reaction chambers, that is those with, for example, rhombic geometry, are filled with the non-aqueous liquid, for example oil. This is due to the fact that if the non-aqueous liquid were to be added first, a thin layer of oil would form within in the neighbouring (circular) chambers meant to hold the aqueous liquids, thus resulting in difficulty in mixing of beads in the aqueous chamber due to underlining of oil phase.

Any material which is suitable for cell-culture, that is any material that is biocompatible and that does not cross-react with target analytes or any of the liquids used in standard enzyme-linked immunosorbent assay protocols can be used for producing the present invention. Materials from which an ELISA plate is made include, but are not limited to, polystyrene, poly methyl methacrylate, polypropylene, polycarbonate, glass and combinations thereof. In one example, the ELISA plate is made from poly methyl methacrylate.

As previously described, the ELISA plate disclosed herein comprises a plurality of reaction chambers. These reaction chambers are located on the plate in which the chemical or biochemical reactions, for example of the enzyme-linked immunosorbent assay, take place. Therefore, the size of the reaction chambers is dictated by the volume required for an ELISA assay to be performed. A typical ELISA assay can have a sample volume of between 50 μl to 150 μl, between 175 μl to 200 μL or between 180 μl to 200 μl. In one example, as disclosed herein, the volume of the reaction chambers is selected from about 50 μl, about 60 μl, about 70 μl, about 80 μl, or about 90 μl. In another example, the volume of the reaction chambers is 70 μl to 80 μl.

The reaction chambers, as described herein, can take form of any suitable shape. The geometry of the reaction chamber is dictated only by the practicality of the form for actuation of the samples between the reaction chambers. Thus, in one example, the plurality of reaction chambers can comprise a single geometry or multiple geometries. In another example, the reaction chambers comprise two geometries. In another example, the reaction chambers comprise three or more geometries. A possible use for the different geometries is, for example, the optical differentiation between the different reaction chambers and their various contents. For example, it is possible to have all reaction chambers holding non-aqueous solutions to be of one geometry, and the remaining reaction chambers on a plate to have a different geometry. Instead of using different geometries to denote the different content in the reaction chambers, this optical differentiation of the different reaction chambers can also be done using markings (coloured or not) on the top or side of the ELISA plate, or colouring the sides of the plate. Having said that, in one example, a circular chamber is used for aqueous liquids, as it provides uniformity in the shape of the reaction chamber and in mixing which can take place in such a reaction chamber, with equal surface tension along the edges. Also, as discussed in another section of the disclosure, for example, an opening in a circular reaction chamber provides easy access for the beads to transfer to, for example, an adjacent rhombic chamber. On the other hand, in one example, the shape of the non-aqueous reaction chamber is can be, but is not limited to a square, rectangle, circular or ellipsoid shape.

In one example, the geometries of the reaction chambers comprise, but are not limited to cuboid, cube, cylindrical, circular, round, spherical, rectangular, square, triangular, polygonal, rhombic, hexagonal prism, elliptical, ellipsoid or trapezoidal.

The geometries of the reaction chambers on the ELISA plate, or even in the same row, can be present on the ELISA plate in the form of a recurring pattern, or alternating geometries. The selection of the recurrence or pattern of the reaction chamber geometries is dependent on experimental requirements, for example a number of chambers of one type (for example, chambers holding aqueous wash solutions) next to a single chamber of a different fluid (for example, a chamber holding non-aqueous separating fluids). In one example, the ELISA plate as described herein comprises of reaction chambers in the same row, which comprise a first geometry and a second geometry different from the first geometry. This constellation would result, for example, in a plate that has two round reaction chambers next to each other, followed by a square reaction chamber. Or, the enzyme-linked immunosorbent assay plate can comprise strictly alternating geometries, for example alternating round and square reaction chambers. Thus, in one example, the ELISA plate is as described herein, wherein the first geometry is cylindrical and the second geometry is rhombic. In another example, the reaction chambers are all cylindrical. In yet another example, the reaction chambers are all rhombic.

The enzyme-linked immunosorbent assay plates as described herein are can be produced in strips (that is in rows) or columns, depending on request of the experimenter and the manufacturing capabilities. However, regardless of how the plate is divided, each individual enzyme-linked immunosorbent assay plate comprises a base plate and a top plate. In one example, the base plate is a solid plate. In another example, the top plate comprises perforations forming the reaction chambers. In another one example, the base plate comprises a solid plate and wherein the top plate comprises of perforations forming the reaction chambers. It is also envisioned that the reaction chambers are etched into a solid plate, thereby removing the requirement of the enzyme-linked immunosorbent assay plates needing assembly. In one example, the plate may also be poured into an appropriate mould, thereby making only the top plate or the entire plate as such.

One requirement for ensuring adequate performance of enzyme-linked immunosorbent assays (ELISAs) is to prevent a target analyte from adhering to surfaces other than those enabled for detection of the target analyte, as any binding of the target analyte to surfaces not enabled for detection results in the loss of signal detection, thereby effecting the overall efficacy and/or sensitivity of the performed assay. One way of preventing unintended adherence of target analyte to unintended surfaces is the use of coating materials, which are then used to coat, for example, the inside surfaces of reaction chambers. These coatings can be in the form of, but are not limited to, sprays, foils, films, solutions, emulsions, polymer coatings, dry coating, metallic coatings and combinations thereof. Thus, in one example, the coating is a film. These coatings can be hydrophobic or hydrophilic, depending on the target analyte. If the target analyte is a biological molecule, for example a hydrophobic protein, then the coating to prevent adhesion of said hydrophobic protein to the surface of the reaction chamber would be a hydrophilic coating. Thus, in one example, the enzyme-linked immunosorbent assay plate as disclosed herein comprises a hydrophobic layer. The location of the coating for preventing unintended adhesion can be found on all surfaces of the reaction chamber. The coating can alternatively be found only on the wall of the reaction chamber. The coating can also be found only on the base of the reaction chamber. In one example, the coating is disposed between the base plate and the top plate.

An alternative to having a target analyte bind to the surface of the reaction chamber, or in cases where, for example, the concentration of the target analyte in the sample is limited and possible pre-concentration of the target analyte is required, carriers, including but not limited to magnetic beads, magnetic particles, superparamagnetic beads or particles, polymer-coated magnet core beads or particles and the like, the difference between beads and particles in general being that particles have an irregular surface, whereas beads have an predominantly round surface. These carriers may be made of substances that enable the capture of the target analyte according to various physical or chemical principles. For example, when the carrier is made of sepharose, it is possible to include pores in the surface of the carrier and thereby concentrate only analytes of a specific molecular size. In another example, the carrier is coated with, for example, an analyte-capturing antibody, thereby specifically concentrating and binding only the antigens (that is the target analyte) of said antibody. In one example, magnetic beads are used. In yet another example, the magnetic beads are coated with target analyte-specific antibodies. For example, if the target analyte is the 38 kDa tuberculosis protein, then the antibody used is anti-38 kDa. As antibodies are known to be extremely specific for their corresponding antigens, it is also possible to coat carriers with more than one antibody. In another example, the carrier is coated with the antigen. This approach is used when the target analyte is an antibody, for example, when detection the strength of an immune reaction to an infectious particle or for detecting latent disease or infection. Thus, for example, the carrier is coated with an antigen and the resulting signal generated after the corresponding ELISA has been performed would inform a person skilled in the art if an infection or a disease is present. The results of the performed ELISA also indicate to the person skilled in the art whether the infection or disease is active (that is the infectious agent is for example actively replicating), latent (that is the infectious agent is replicating at a level that is considered to be below the defined threshold for an active infection; this latent infection can be present in an asymptomatic fashion), dormant (that is the presence of the infectious agent has been determined but the infectious agent is not replicating) and non-existent (that is there is no infectious agent present in the subject).

In the event that the carrier utilised in the present invention is magnetic in nature, it is required that a further magnet be present in order to move the carrier from one reaction chamber to the next. Thus, in one example, a further magnet capable of actuating, that is moving, the magnetic beads is present. The polarity of said further magnet must be so that it is capable of magnetically attracting the magnetic beads within the reaction chambers through the walls of the reaction chambers, so that the magnetic beads do not come in contact with the further magnet during sample handling and moving. The size of the ELISA plate used in the invention dictates the size of the further magnet required for sample handling. Therefore, the size of the magnet includes, but is not limited to, between 10 mm to 150 mm, between 100 mm to 200 mm, about 25 mm, about 35 mm, about 50 mm, about 55 mm, about 60 mm, about 85 mm, about 90 mm, about 105 mm, about 150 mm or about 180 mm. Thus, if for example, the width of the ELISA plate (that is the length along a column of reaction chambers) is 50 mm, then the length of the further magnet is at least 5 mm. It is also possible to use one or more magnets for moving said magnetic beads simultaneously. It is also possible to move part of the magnetic beads first using one magnet, and using that same magnet to go back and move magnetic beads in other reaction chambers. As used herein, the term “width” is defined as the measurement along one external edge of the plate, wherein the width measurement is shorter than the length measurement. In one example, the length of the magnet is 70 mm.

The size of the reaction chambers further dictates the overall size of the ELISA plate as described herein. Alternatively, it can be said that the size of the ELISA plate dictates the volume of the reaction chambers. The size of the ELISA plate is scalable and includes, but is not limited to for example, micro-scale or nanoscale. In absolute terms, the size of the ELISA plate includes, but is not limited to, a length of between 25 mm to 100 mm, between 30 mm to 150 mm, between 80 mm to 200 mm, about 60 mm, about 80 mm, about 90 mm, about 120 mm, about 130 mm or about 150 mm, whereby the length of the ELISA plate is defined as the longest edge of the plate, that is the length measurement is by definition longer than that of the width measurement. In one example, the length of the ELISA plate is 95 mm. Accordingly, the width will be adjusted when required along with the adjustment of the length of the plate.

The size of the reaction chambers is also influenced by the height of the ELISA plate used. An ELISA plate with a low height will not be able to yield enough reaction chambers for performing the requisite ELISA, as the resulting reaction chambers may be too wide to all fit on the surface of the plate. Therefore, the height of the ELISA plate includes, but is not limited to, a total height of between 1 mm to 10 mm, between 5 mm to 25 mm, between 15 mm to 20 mm, about 14 mm, about 15 mm, about 17 mm, about 19 mm or about 20 mm. Thus, in one example, the height of the ELISA plate in total is 5 mm. This in turn means that, for example, if an ELISA plate is constructed of more than one piece, for example a base plate and a top plate, the combined height of both plates is also 5 mm. Another consideration to be had us that if upon completion of the experiment, the ELISA plate is to be read in a standard spectrometer or a standard plate reader, these instruments have sizing which need to be adhered to, lest otherwise the measurements be distorted or not adequately compensated for.

Another consideration is that the opening at the top of the reaction chamber is not to be larger than technically necessary, as a larger reaction chamber opening will result in fluids contained within these chambers to evaporate at a faster rate than if the reaction chamber has a small opening. Having said that, a small opening can result in the sample not being loaded into the reaction chambers properly if, for example, correctly sized pipettes or needles are not available. All these factors affect the sensitivity and accuracy of the performed ELISA by influencing the outcome of the ELISA. Also considered is the use of a cover on top of the ELISA plate, thereby preventing any excess loss of reaction fluids or sample via evaporation.

The various parts of the ELISA plate, as described herein, can also be described as a system for detecting a target analyte comprising an enzyme-linked immunosorbent assay (ELISA) plate as disclosed herein, a plurality of magnetic beads and a magnet configured to cooperate with the magnetic beads. In one example, the magnet of the system is configured to control the magnetic beads to move between the reaction chambers in the same row. The system can further include, but is not limited to, a detector for detecting a signal generated from the reaction chambers at the end of the ELISA reaction.

Also disclosed herein is a kit comprising of an enzyme-linked immunosorbent assay (ELISA) plate as described herein, a plurality of magnetic beads and a magnet. In one example, the plate is a microplate.

One example of the use of the claimed invention is performing an enzyme-linked immunosorbent assay (ELISA) using a system as described herein. The liquids used to perform the ELISA, as described herein are chosen based on principles and requirements of an ELISA known in the art. For example, paring and combination of the capture, primary and secondary antibodies is performed according to established principles known in the art. The detection substrate and detection solution required for signal generation at the end of an ELISA are chosen according to requirements and available means for measuring said signal. The types of detection methods include, but are not limited to, colorimetric, luminescent, bioluminescent, fluorescent, photometric, and radiographic. In one example, the reaction chambers of the ELISA plate are liquid-filled. In another example, the reaction chambers, or cells, of the columns of the ELISA plate are filled with alternating liquids. In yet another example, the liquids are either aqueous or non-aqueous.

In yet another example, a sample comprising a target analyte is incubated with a plurality of magnetic beads capable of capturing said target analyte in the first chamber of the ELISA plate according to any of the preceding claims. In another example, a sample comprising one or more target analytes is incubated with a plurality of magnetic beads capable of capturing said one or more target analytes in the first chamber of the ELISA plate according to any of the preceding claims. In yet another example, the plurality of magnetic beads is moved from the first reaction chambers to subsequent reaction chambers using the magnet. In a further example, the plurality of magnetic beads is incubated in subsequent reaction chambers. In one example, previous steps of incubating and moving the plurality of magnetic beads are repeated multiple times. In another example, the steps are repeated until the final chamber in the row is reached. In yet another example, the signal generated in the final reaction chamber is detected using an appropriate means. In another example, one or more intermediate measurements are made between the first reaction chamber and the final reaction chamber. It is noted that any one or more of the previously outlined steps may be performed repeatedly and in any combination with each other according to the experimenter's requirements. In a further example, an enzyme-linked immunosorbent assay (ELISA) is performed using a system as described herein, wherein the reaction chambers of the ELISA plate are liquid-filled, the method comprising (a) loading the cells of the columns of the ELISA plate with alternating liquids, wherein the liquids are either aqueous or non-aqueous; (b) incubating a sample comprising one or more target analytes with a plurality of magnetic beads capable of capturing said one or more target analytes in the first chamber of the ELISA plate according to any of the preceding claims; (c) moving the plurality of magnetic beads from the first reaction chambers to subsequent reaction chambers by using the magnet; (d) incubating the plurality of magnetic beads in subsequent reaction chambers; (e) repeating steps (c) to (d) until the final chamber in the row is reached; and (f) detecting the signal generated in the final reaction chamber.

Also described herein is method of performing an enzyme-linked immunosorbent assay (ELISA) using a system as described herein. In one example, the method comprises incubating a sample comprising one or more target analytes with a plurality of magnetic beads capable of capturing said one or more target analytes in the first chamber of each row of the ELISA plate as described herein; loading the subsequent reaction chambers of the columns of the ELISA plate with alternating liquids, wherein the liquids are either aqueous or non-aqueous; moving the plurality of magnetic beads from the first reaction chambers of each row to subsequent reaction chambers of the same row by using the magnet; incubating the plurality of magnetic beads in subsequent reaction chambers; repeating the previous steps until the final chamber in the row is reached; and detecting the signal generated in the final reaction chamber. This method can include, for example, one or more intermediate measurements are made between the first reaction chamber and the final reaction chamber of each row. This method can also include that the reaction chambers are filled with alternating liquids.

The liquids utilised in the present invention can be, but are not limited to, non-aqueous and aqueous liquids. In one example, the non-aqueous liquid is a non-polar liquid. The function of the non-aqueous solution is to act as a barrier between the reaction chambers which contain aqueous solutions, thereby preventing mixing or dilution of the various aqueous solutions. Also, the non-aqueous solutions act as a fluid impermeable barrier which, in spite of being fluid impermeable, enables the movement of magnetic beads into and out of connected reaction chambers without the aqueous solution being carried over. Therefore, the non-aqueous solutions must be viscous enough to be able to stay within their allotted reaction chambers and thereby prevent the aqueous solution in the neighbouring chambers to flow in, but also must be fluid enough to allow the magnetic beads to pass through the non-aqueous liquid between chambers. Thus, in another example, the non-polar liquid includes, but is not limited to, mineral oil, silicone oil, linseed oil, sunflower oil, rapeseed oil and paraffin.

The aqueous solutions utilised in the present invention are the standard solutions known and used in the art for performing an enzyme-linked immunosorbent assay (ELISA). The function of a wash buffer, for example, is to wash off residual molecules from the magnetic beads after each reaction step, thereby preventing carryover which in itself can cause false positive results. The function of the primary antibody is to positively identify the presence of the intended target analyte on the magnetic bead, whereas the secondary, conjugated antibody serves, for example, the dual purpose of amplifying the signal of the target analyte and converting the binding of said secondary antibody into a readable signal for detection, for example, using a detection solution, downstream. Thus, in one example, aqueous liquid is selected from the group consisting of a wash buffer, a primary antibody solution, a secondary antibody solution, an enzyme solution and a detection solution. In another example, the detection solution includes, but is not limited to, colorimetric, luminescent, bioluminescent, fluorescent and radiographic solutions.

The comparison of the concentration of one or more target analytes, or the determination of the presence or absence of one or more target analytes (for example, one or more proteins, oligomers or oligonucleotides), in a subject is determined based on the strength of the signal generated by the enzyme-linked immunosorbent assay (ELISA) reaction as described herein. Generally speaking, a comparison is based on the comparison of the level of one or more target analytes determined in the subject and the level of the same one or more target analytes determined in a control group or control individual. In the present disclosure, the control subject or individual is an individual that is disease-free. That is, the control individual is an individual that is free of the disease for which the test is undertaken. Usually, the term disease-free implies that the subject is healthy.

In one example, the disease for which the test is being undertaken is tuberculosis. However, the presence of any other disease can be determined using the invention provided herein, so long as appropriate analytes with sufficient sensitivity and accuracy have been identified. In one example, the microchip enzyme-linked immunosorbent assay disclosed herein utilises one or more target analytes that are specific to the identification of, for example, a tuberculosis infection. A target analyte can but is not limited to, an antigen, an antibody, a protein, an oligonucleotide, a nucleic acid sequence, a polypeptide and combinations thereof. Capture and detection antibodies required by the ELISA need to be adjusted according to the target analyte in line with principles and concepts known in the art. In one example, the target analyte includes, but is not limited to, a Mycobacterium tuberculosis (Mtb) surface glycolipid i.e. trehalose 6,6′-dimycolate (TDM), a 38 kDa glycolipoprotein and antigen 85A (Ag85A). The latter, 38 kDa glycolipoprotein and antigen 85A (Ag85A), are two purified culture filtrate proteins which are considered to be antigens based on their known immunogenicity and their application in tuberculosis serodiagnosis. The ELISA relies on the actuation of antigen-coated magnetic beads through sequentially organised reagents for capturing Mycobacterium tuberculosis antigen-specific IgG from the plasma, followed by labelling and colorimetric detection. ELISAs, for example ones used especially for the detection of tuberculosis, featuring detection of anti-trehalose 6,6′-dimycolate (anti-TDM) IgG response showed significantly higher sensitivity (72%) compared to sputum smear microscopy (56%) and comparable sensitivity to standard culture tests (78%) for differentiating ATB patients from healthy control (HC) individuals.

As known in the art, a standard enzyme-linked immunosorbent assay (ELISA) can take anywhere from 3 hours to 6 hours, to overnight, depending on the specificity of the antibodies and the quality of the detection substrate used. Typically, a single analyte ELISA takes about 3 hours from start to completion, longer if prior incubation of the sample with the ELISA plate is required, that is the binding of the target analyte from the sample to the surface of the reaction chamber via, for example, hydrophobic interaction. The invention, as described herein, results in a substantial time saving compare to known ELISAs in the art, as with the present inventions, an ELISA can be completed within 15 minutes, from sample addition to detection. This 15 minute mark is also the minimum specification required for point-of-care tuberculosis testing as defined by the World Health Organisation (WHO).

As used herein, the term “sample” includes, but is not limited to, any quantity of a substance from a living thing or formerly living thing. Such living things include, but are not limited to, humans, mice, monkeys, rats, rabbits, dogs, pigs and other animals. Such substances include, but are not limited to, serum, blood, whole blood, blood plasma, serum, phlegm, sweat, stool, urine, sperm, cells, organs, tissues, bone, bone marrow, lymph, lymph nodes, synovial tissue, endothelial cells and skin. Also included therein are laboratory samples, for example, but not limited to, biopsy samples, passaged cells, cell culture samples, cell culture supernatant and lysed cells.

The ELISA as described herein provides a simple sample addition (sample-in) to colorimetric detection (answer-out)-based system to differentiate, for example, active tuberculosis from healthy controls/latent tuberculosis infections with results available in less than 15 minutes. The higher sensitivity of, for example, trehalose 6,6′-dimycolate (TDM)-based ELISAs compared to sputum microscopy (72% vs. 56%), is useful for rapid screening and diagnosis of individuals with clinically active disease. This ultrafast test is simple and inexpensive, well suited to performing an initial screen of potential patients who can later be verified using, for example, PCR-based assays or other methods known in the art. The sensitivity of, for example trehalose 6,6′-dimycolate (TDM)-based ELISAs (72%) is in close agreement to classical trehalose 6,6′-dimycolate (TDM) enzyme-linked immunosorbent assays (69%, 95% confidence interval (CI) 28-94%). Similarly, the sensitivity of, for example, the 38 kDa-based ELISA (46%) in smear-positive samples is in close agreement to that observed in studies using plate-bound ELISAs (47%, 95% confidence interval (CI), 39-55%). The ELISA test, for example the ELISA tests performed for tuberculosis, performs well in comparison to other commercial tests that use, for example 38 kDa and TDM as antigens, such as Pathozyme Myco-G (Omega diagnostics, UK) (10-85% sensitivity) and the tuberculosis glycolipid assay (Kyowa Medex, Japan) (59-90% sensitivity), respectively. However, the ELISA plate and the ELISA method as disclosed herein offer the advantages of being faster, simpler, and cheaper.

The ELISA platform, as disclosed herein, has three important technological implementations compared with a conventional enzyme-linked immunosorbent assay. First, the working principle of ELISA, i.e. magnetic bead actuation through chambers pre-filled with stationary reagents, circumvents the need for additional equipment, such as expensive pumps, valves, and sample metering which usually require, for example, challenging micro-scale fabrication techniques. The ELISA plate requires simple non-lithographic fabrication methods, and no sophisticated tools are needed to perform the test. Thus, the cost of the test, including reagents and device fabrication, remains extremely low (less than U.S. $10 per test). Second, contrary to the conventional enzyme-linked immunosorbent assays (ELISAs), the ELISA plate and approach as described herein utilises polymeric horseradish peroxidase (HRP) labels as opposed to single HRP-labelled secondary antibodies. The higher ratio of horseradish peroxidase to polymer in the labelling step (400 horseradish peroxidases per polymer) amplifies the colorimetric signal and enables detection of, for example, low IgG titre samples. Third, the bead-based enzyme-linked immunosorbent assay employed in the microchip has specific advantages over conventional flat surfaces. The high local concentration of antigens bound on the bead surface promotes efficient antigen-antibody binding compared to flat plate surfaces, where binding is facilitated by slow diffusion, and requires several hours to reach saturation. This translates to faster reaction times for each step in the ELISA as disclosed herein, significantly reducing the overall time of the assay to about 15 minutes using plasma sample matrices, but still performing as efficiently as the standard classical enzyme-linked immunosorbent assay. Moreover, the plasma-based testing presented herein is easy to perform because of its simple sample collection (whole blood, venous/finger prick) compared to sputum sample (deep cough, several attempts required). This is particularly relevant for, for example, sample collection from children, for whom sputum collection is challenging and invasive.

Furthermore, the current ELISA plate and assay, as disclosed herein, has many advantages over commercial immunochromatographic (IC) tests, which are widely used for, for example tuberculosis diagnosis in low-income, high burden countries. Although immunochromatographic tests are user-friendly, rapid, and affordable, they lack the sensitivity of a classical enzyme-linked immunosorbent assay (53%, 95% confidence interval (CI) 42-64%) and the results are qualitative, relying on a subjective interpretation of aggregated gold nanoparticle band intensity. Furthermore, immunochromatographic tests are only able to provide binary reports (yes/no) for a single antigen, and analysing responses against multiple antigens is complex. In contrast, the ELISA device as disclosed herein represents a simple and robust platform, with sensitivity comparable to a bench-top enzyme-linked immunosorbent assay and provides an accurate numerical interpretation of responses against multiple biomarkers.

The ELISA platform as disclosed herein is a flexible technology that can be adapted to diagnosis of other diseases. Simultaneous use of multiple rows could even be used to detect multiple biomarkers for several diseases at the same time. The detection mode is not restricted to a colorimetric readout, and can be translated to more sensitive, but not limited to, electrochemical, electro-chemiluminescent, chemiluminescent, fluorescent, and plasmonic readouts. The current invention has the applicability to be integrated into a fully automated device for operation in resource-limited settings. It is envisaged that the automation of magnet actuation and the integration of a portable colorimetric sensor into a single device presented here will provide a single-step miniaturised assay for disease identification and detection, in one example for tuberculosis detection. Such a device is truly useful in point-of-care of, for example, tuberculosis diagnosis for screening patients and tuberculosis contacts, where immediate treatment decisions are of high clinical significance.

The current ELISA plate as disclosed herein has several notable advantages over currently available tests, i.e. the device is fabricated with simple non-photolithographic methods, is miniaturised, and does not require sophisticated tools to perform the assay. The assay platform is affordable, costing under U.S. $10 per test. The assay is ultrafast and can be completed within 15 min, and the performance of the ELISA assay using the plate as disclosed herein is equivalent to gold standard tests. Also, the ELISA assay using the plate as disclosed herein is easily implemented in combination with sputum microscopy to speed up tuberculosis diagnosis in triage and community settings.

Thus, disclosed herein is a simple and low-cost serodiagnostic test, for example for detecting tuberculosis, based on a microchip enzyme-linked immunosorbent assay platform for the detection of, for example, anti-mycobacterial IgG in plasma samples in less than 15 minutes is reported. The ELISA plate as disclosed herein employs a flow-less, magnet-actuated, bead-based enzyme-linked immunosorbent assay for simultaneous detection of one or more IgG responses against multiple mycobacterial antigens. Anti-trehalose 6,6′-dimycolate (TDM) IgG responses were the strongest predictor for differentiating active tuberculosis (ATB) from healthy controls (HC) and latent tuberculosis infections (LTBI). The trehalose 6,6′-dimycolate (TDM)-based ELISA assay demonstrated superior sensitivity compared to sputum microscopy (72% vs. 56%) with 80% and 63% positivity among smear-positive and smear-negative confirmed active tuberculosis infection (ATB) samples, respectively. Receiver operating characteristic analysis indicated good accuracy for differentiating ATB from HC (AUC=0.77). Thus, TDM-based ELISA assays can be used as a screening device for rapid diagnosis at the point-of-care.

As disclosed herein, in one example, the invention comprises of a microchip ELISA for the rapid serodiagnosis of Mycobacterium tuberculosis infection. The point-of-care test (POCT) detects the presence of human immunoglobulin (hIgG) against Mycobacterium tuberculosis specific antigens. The microchip enzyme-linked immunosorbent assay platform is a rapid test that can discriminate between individuals with active tuberculosis disease (ATB), latent tuberculosis disease (LTB) and healthy individuals, with results available within fifteen minutes.

In another example, a microchip-based immunoassay for the simultaneous detection of human IgG antibody response to multiple Mycobacterium tuberculosis lipid (trehalose 6, 6′-dimycolate; TDM) and tuberculosis protein antigens (for example, 38 kDa and Antigen 85A) is described herein.

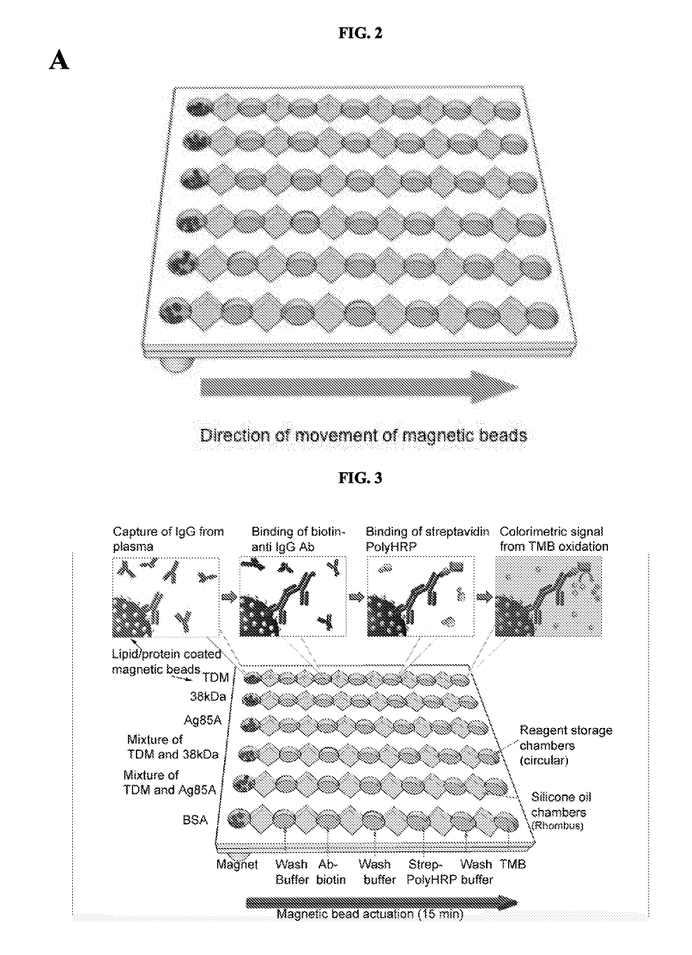

The microchip device consists of multiple, sequentially organized, aqueous (circular) chambers for reagent storage such as wash buffers, dispersion of magnetic beads (MB), biotin-labelled antibodies, streptavidin polymeric enzyme labels and colorimetric substrate (FIG. 2B). The aqueous chambers were separated by an immiscible silicone oil phase (rhombus chambers) to prevent mixing of reagents. The microchip immunoassay employs magnet assisted movement of antigen coated magnetic beads through spatially arranged aqueous reagent phase and an oil phase for plasma incubation, washing, labelling and detection steps. The device features six channels, with each channel containing either individual or mixture of Mycobacterium tuberculosis lipids and protein antigen coated magnetic beads. For example, channel 1 contained TDM coated magnetic beads, while channel 4 contained a mixture of TDM coated magnetic beads and antigen 85A coated magnetic beads. Magnetic beads were then simultaneously actuated from an aqueous phase to oil and back into an aqueous phase using six magnets position beneath the microchip.

Disclosed herein is also a method of detecting one to more target analytes in a sample. These one or more target analytes include, but are not limited to components of the mammalian immune system, for example antibodies and cytokines. In one example, the target analyte is a cytokine. Examples of cytokines include, but are not limited to chemokines, interferons, interleukins, lymphokines and tumour necrosis factors. Examples of chemokines include, but are not limited to C chemokines, CC chemokines, CX3C chemokines and CXC chemokines. Examples of CC chemokines include, but are not limited to CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27 and CCL28. Examples of C chemokines include, but are not limited to XCL1 and XCL 2. Examples of CX3C chemokines include, but are not limited to CX3CL1. Examples of CXC chemokines include, but are not limited to CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 and CXCL17. Examples of interferons include, but are not limited to Interferon alpha, Interferon alpha 1, Interferon alpha 2, Interferon alpha 4, Interferon alpha 5, Interferon alpha 6, Interferon alpha 7, Interferon alpha 8, Interferon alpha 10, Interferon alpha 13, Interferon alpha14, Interferon alpha16, Interferon alpha 17, Interferon alpha 21, Interferon beta 1, Interferon omega, Interferon epsilon 1 and Interferon kappa. Examples of tumour necrosis factors include, but are not limited to, tumour necrosis factor alpha (TNF-α, cachectin), tumour necrosis factor beta (TNF-β), tumour necrosis factor ligand superfamily member 4 (TNFSF4), tumour necrosis factor ligand superfamily member 8 (TNFSF8), tumour necrosis factor ligand superfamily member 9 (TNFSF9), tumour necrosis factor ligand superfamily member 11 (TNFSF11, RANKL), tumour necrosis factor ligand superfamily member 12 (TNFSF12; TWEAK), tumour necrosis factor ligand superfamily member 13 (TNFSF13), tumour necrosis factor ligand superfamily member13 (TNFSF13b), tumour necrosis factor ligand superfamily member 14 (TNFSF14), tumour necrosis factor ligand superfamily member 15 (TNFSF15), tumour necrosis factor ligand superfamily member 18 (TNFSF18), lymphotoxin-alpha (LT-alpha), LTA, LTB, T-cell antigen gp93 (CD40L), CD27L, CD30L, CD70, EDA, FASL, FASLG, 4-1BBL, OX40L, proliferation-inducing ligand (APRIL) and tumour necrosis factor related apoptosis inducing ligand (TRAIL, TNFSF10). Examples of interleukins include, but are not limited to, interleukin 1 (IL-1), interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 1 (IL-8; also known as CXCL8), interleukin 9 (IL-9), interleukin 10 (IL-10), interleukin 11 (IL-11), interleukin 12 (IL-12), interleukin 13 (IL-13), interleukin 14 (IL-14), interleukin 15 (IL-15), interleukin 16 (IL-16), interleukin 17 (IL-17), interleukin 18 (IL-18), interleukin 19 (IL-19), interleukin 20 (IL-20), interleukin 21 (IL-21), interleukin 22 (IL-22), interleukin 23 (IL-23), interleukin 24 (IL-24), interleukin 25 (IL-25), interleukin 26 (IL-26), interleukin 27 (IL-27), interleukin 28 (IL-28), interleukin 29 (IL-29), interleukin 30 (IL-30), interleukin 31 (IL-31), interleukin 32 (IL-32), interleukin 33 (IL-33), interleukin 35 (IL-35), and interleukin 36 (IL-36). In another example, the target analyte is an antibody.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Chemicals and Materials.

Superparamagnetic microbeads, for example Tosyl-activated superparamagnetic microbeads (MBs, Dynabeads, Cat. no. 14013, 4.5 μm or 1.0 μm diameter, 4×10⁸ beads/ml, 30 mg/ml), were from Invitrogen. Trehalose 6,6′-dimycolate from Mycobacterium bovis (TDM, Cat. no. T3034), and fatty acid-free bovine serum albumin (BSA, Cat. no. A7030) were from Sigma. Goat F(ab′)₂ anti-human IgG (H+L) labelled with biotin was from Southern Biotech. Nunc PolySorp 96-well plates, 1-step ultra TMB ELISA, and Pierce streptavidin poly-HRP were obtained from Thermo Scientific. Mycobacterial recombinant 38 kDa and Ag85A proteins were obtained from Mybiosource (CA, USA). Magnets, for example neodymium disc magnets (diameter 5 mm, thickness 2 mm), were from AliExpress Global Retail. Unless otherwise specified, all experiments were performed using PBS buffer without Ca⁺² and Mg⁺² ions.

Device Fabrication.

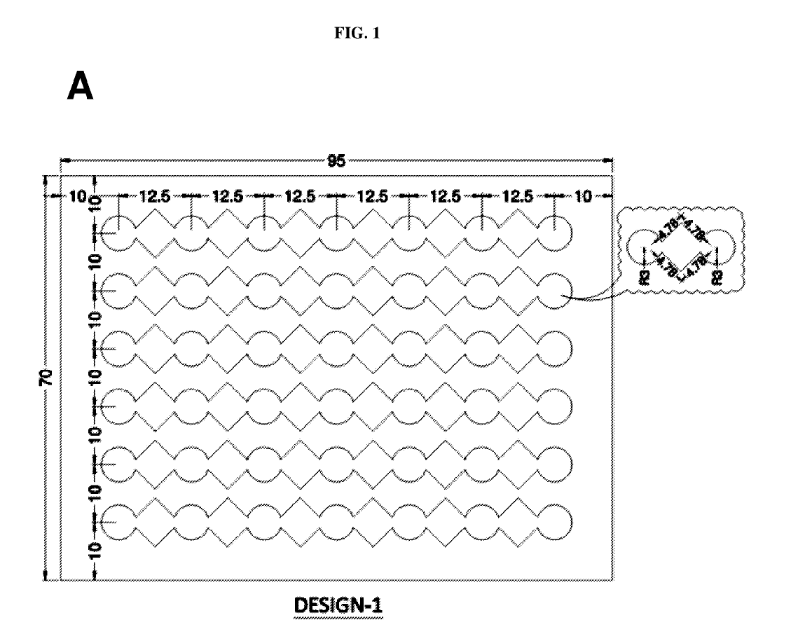

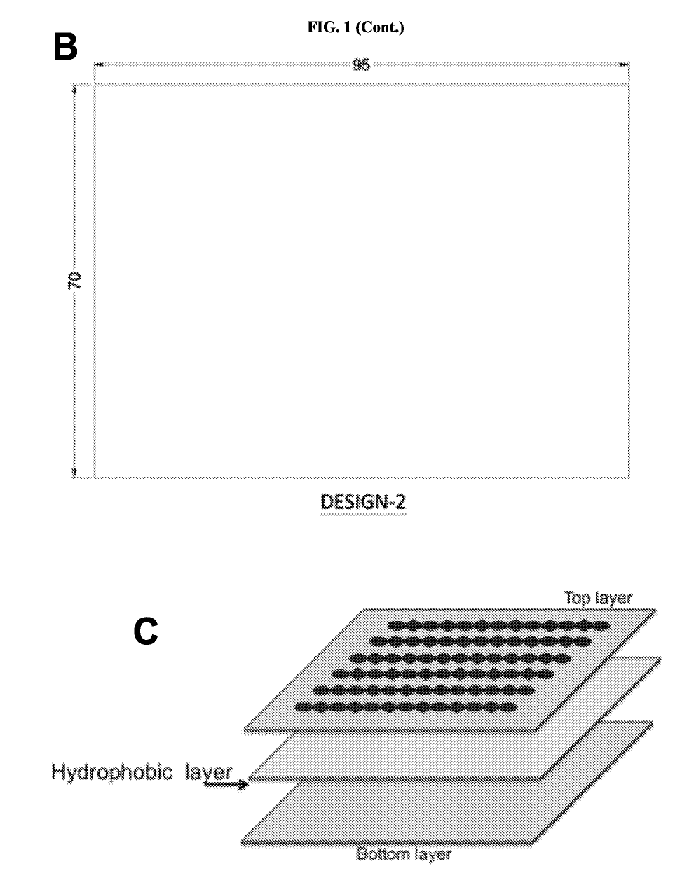

The microchip, or the ELISA plate as disclosed herein, was fabricated using a non-lithographic technique as reported in prior art. The device consisted of two clear poly(methyl methacrylate) (PMMA) laser cut sheets assembled and bonded (also referred as, for example, a “microchip”) or two PMMA laser cut sheets with a parafilm layer between the sheets assembled and bonded (referred as “parafilm bonded microchip”). The templates were designed using AutoCAD software. The designs were then supplied to vendor (Ying Kwang Acrylic Trading, Singapore) for laser cutting services. The top template (FIG. 1A) was laser cut on 3-mm thick PMMA, whereas the bottom template (FIG. 1B) was cut on 1.5-mm thick PMMA. The two templates or the two templates and a parafilm layer (FIG. 1C) were bonded using spray adhesive (3M, Super 75). The adhesive was allowed to bond and dry for 20 min before the microchip was ready to use. The approximate dimensions of the six-channel microchip device were 95 mm×70 mm×5 mm (length×breadth×height).

Human Subjects and Sample Collection.

Blood samples of active tuberculosis (ATB), healthy controls (HC), and latent tuberculosis infections (LTBI) individuals were collected at the Tuberculosis Control Unit, Tan Tock Seng Hospital (TTSH), Singapore. Blood plasma was separated in a BSL3 facility, and immediately stored at −80° C. Of the samples collected, plasma from 65 ATB, 41 HC, and 40 LTBI individuals were randomly selected and stratified based on clinical data, such as the interferon-gamma release assay, sputum smear microscopy, and bacterial culture tests. The study was approved by institutional IRB (NHG DSRB No. 2010/00566). Sputum samples were stratified based on the AFB (Acid fast bacilli) smear grade, which was performed according to the American Thoracic Society (ATS) with negative (−ve) representing 0 AFB/100 fields; 1+ representing 1-9 AFB/100 fields; 2+ representing 1-9 AFB/10 fields; 3+ representing 1-10 AFB/field and 4+ representing>10 AFB/field.

Sample Collection for IFN-γ Response in Blood Plasma.

Blood samples of 4 LTBI and 5 HC individuals were obtained in IGRA tubes, which are part of the Quantiferon TB gold assay. One is positive control tube, one is negative control tube and one is test tube (has Mtb specific antigens). In short, blood is collected in these tubes. After overnight incubation IFN gamma is estimated in the plasma from all three tubes using standard 96 well ELISA. In the present setting, blood was drawn in three tubes [Mitogen (positive control tube), Antigen (test tube), and Nil (negative control tube)], in duplicate. These tubes were then incubated overnight at 37° C. The following day, the first set of three tubes was sent to TTSH hospital for the detection of interferon gamma (IFN-γ). In the hospital, this was performed on the separated plasma using standard IFN-γ ELISA (part of the Quantiferon assay). From the second set of three tubes, plasma was separated and used to detect IFN-γ using microchip ELISA method.

Preparation of Lipid and Protein-Coated MBs.

For TDM coating, 0.8 ml of MB stock (4×10⁸ beads/ml) were taken in a glass tube with a screw cap, and magnetically washed sequentially with 70% and 100% ethanol. The MBs were then air dried, and 84 μg TDM in 2.4 ml of solvent (9:1 hexane:ethanol) was added to the dried MBs. The MB dispersion was then sonicated for approximately 1 hour in a water bath until the solvent evaporated to dryness. Control beads (beads without TDM) were similarly sonicated with TDM-free solvent, until the solvent evaporated. This step was followed by chemical and physical blocking, whereby dried TDM-coated MBs were sonicated for 2 min with 1.6 ml of 0.1% BSA/PBS buffer, and subsequently sonicated for another 2 min after addition of 6.4 ml of 0.2 M Tris buffer, pH 8.0. The bead dispersion was then mixed in a slow tilt rotor for 24 h at room temperature and washed with 10 ml of 0.1% BSA four times before reconstituting in 1.6 ml of 0.1% BSA/PBS buffer, and stored at 4° C. until further use.

For protein or antibody coating, 38 kDa glycolipoprotein (38 kDa), antigen 85A (Ag85A) proteins, or mouse anti-human interferon gamma antibody (anti-human IFN-γ Ab, BD #551221), or mouse anti-human IL-2 antibody (anti-human IL2 Ab, BD #555051), or mouse anti-human tumour necrosis factor alpha antibody (anti-TNF-α Ab, BD #551220) were covalently linked to magnetic beads according to the manufacturer's protocol (Invitrogen). Briefly, 0.2 ml of stock MBs were washed with 1 ml of 0.1 M borate buffer, pH 9.5. Then, 40 μg of protein per mg MBs were added and allowed to mix in a slow tilt rotor for 30 min. In the next step, BSA was added to final concentration of 0.1% and allowed to mix for additional 24 h. The MBs were washed twice with 0.1% BSA in PBS, chemically and physically blocked with 0.2 M Tris and 0.1% BSA buffer overnight. MBs were washed twice with 0.1% BSA/PBS, reconstituted in 0.4 ml of 0.1% BSA, and stored at 4° C. until further use.

Characterisation of TDM-Coated Magnetic Beads.

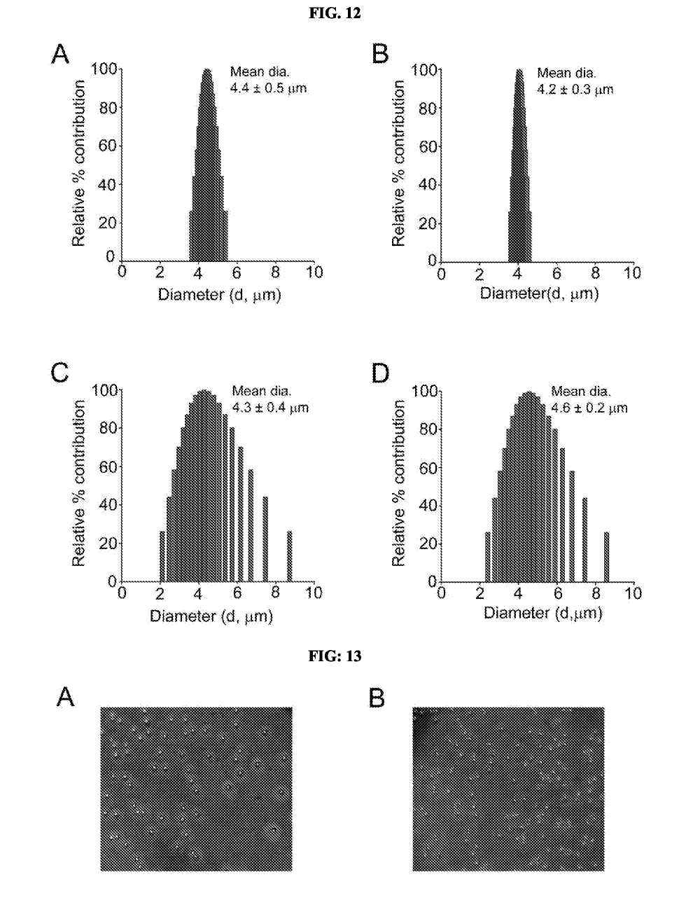

Uniform coating of TDM onto a hydrophobic (magnetic) bead depends on the nominal TDM surface concentration, which is equivalent to the amount of TDM added per total surface area of beads used in the coating process. Because high lipid concentrations lead to the formation of large bead aggregates, different TDM surface concentrations were first assessed (0.16 μg/cm², 0.41 μg/cm², and 0.65 μg/cm²) for optimal coating of TDM onto the beads. The resultant TDM-coated magnetic beads were then tested for size distribution, amount of bound TDM, and the ability to detect anti-TDM IgG using a pooled plasma sample of ATB individuals. Dynamic light scattering (DLS) showed uniform size distribution of magnetic beads irrespective of TDM surface concentrations (FIG. 12). A poly-dispersity index (PI) of <0.21 was obtained for all the MB preparations, indicating the presence of mono-disperse magnetic beads (based on the criteria, PI<0.3 for mono-disperse beads). The mean diameter of the TDM-coated magnetic beads obtained by dynamic light scattering ranged from 4.2-4.6 μm, which was well within the range of uncoated nascent magnetic beads (diameter 4.4 μm). Furthermore, large multi-bead aggregates were not visible by light microscopy, indicating the mono-disperse nature of the TDM-coated magnetic beads (FIG. 13).

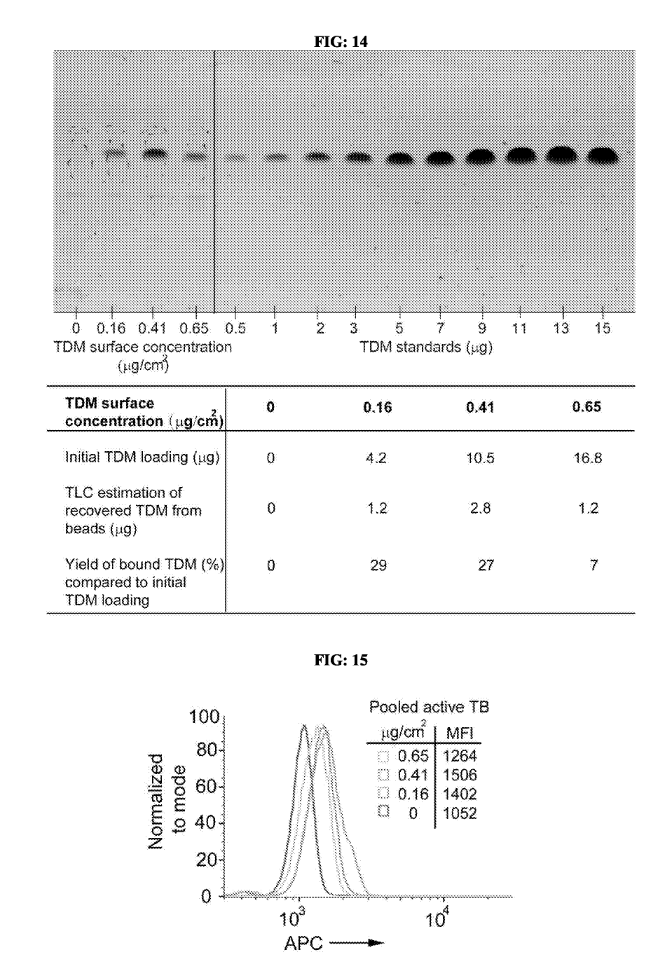

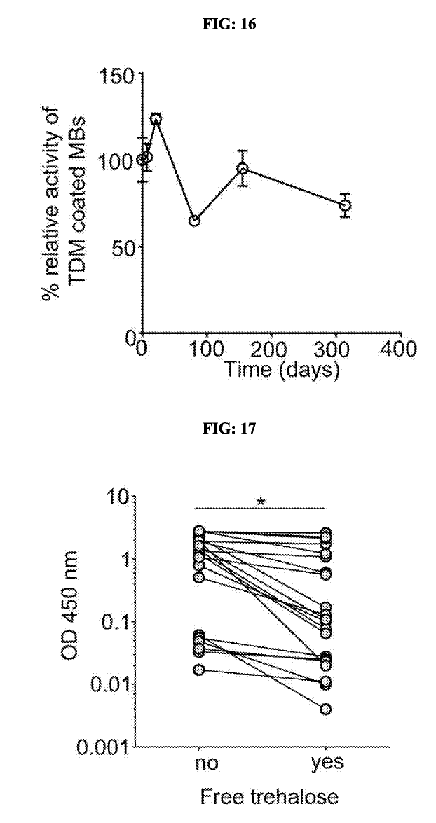

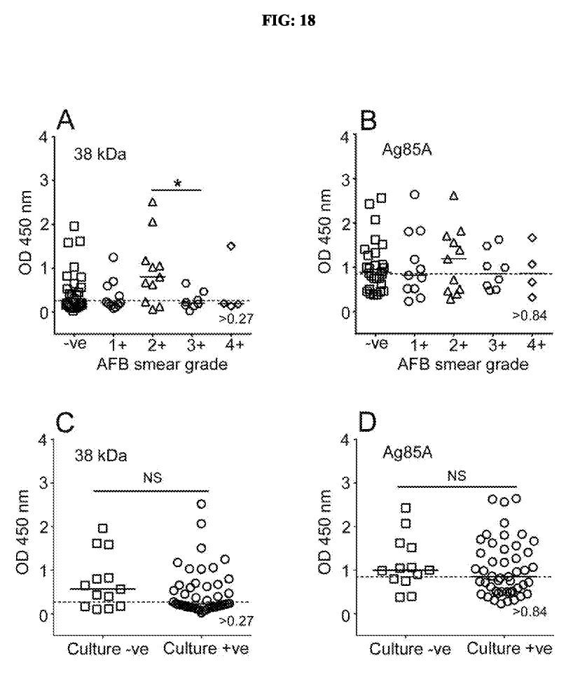

To assess the amount of TDM adsorbed onto the magnetic bead surface, TDM bound to each of the magnetic bead preparations (see above) was extracted for thin layer chromatography (TLC) analysis. Thin layer chromatography analysis indicated that the recovery of bound TDM (%) from magnetic beads prepared using surface TDM concentrations of 0.41 μg/cm² and 0.16 μg/cm² were similar but recovery from magnetic beads using concentrations of 0.65 μg/cm² was lower (FIG. 14). Because higher lipid losses due to nonspecific adsorption to the reaction vials were significant in small batch preparations (0.2 ml), the TDM recovery process was repeated using magnetic beads prepared in a large batch (1.6 ml). In the large batch preparation, magnetic beads with a surface TDM concentration of 0.41 μg/cm² showed 54% recovery of bound TDM. At this concentration and based on the molecular weight of TDM (˜2636 g/mol) and the total number of beads used in the preparation, the number of TDM molecules per bead was estimated at ˜3.2×10⁷. This is in concordance with the theoretical estimate of 3.4×10⁷ of TDM molecules per bead based on the surface area of the bead (6.4×10−11 m2, diameter ˜4.5 μm) and the molecular exclusion area of TDM (˜180° A2/molecule), indicating a uniform monolayer of TDM on the bead surface. Next, the ability of TDM-coated magnetic beads to detect an anti-TDM IgG response in pooled plasma of ATB individuals was tested using a flow cytometry-based MB immunoassay. Bead preparations with a surface TDM concentration of 0.41 μg/cm² displayed the highest levels of anti-TDM IgG antibodies in comparison to magnetic beads prepared using 0.16 and 0.65 μg/cm2 of TDM (FIG. 15). Because temperature variation and reagent storage can influence the performance of an enzyme-linked immunosorbent assay, the stability of TDM-coated magnetic beads (0.41 μg/cm²) was then tested. The magnetic beads maintained 95% activity after 5 months of storage at room temperature and 74% activity after 10 months with a relative variation of 65-125% over time (FIG. 16). Based on the above-mentioned analysis, the magnetic bead preparation with a surface TDM concentration of 0.41 μg/cm² was selected for further use in the magnetic bead ELISA and TDM-based microchip ELISA.

Microchip ELISA Design.

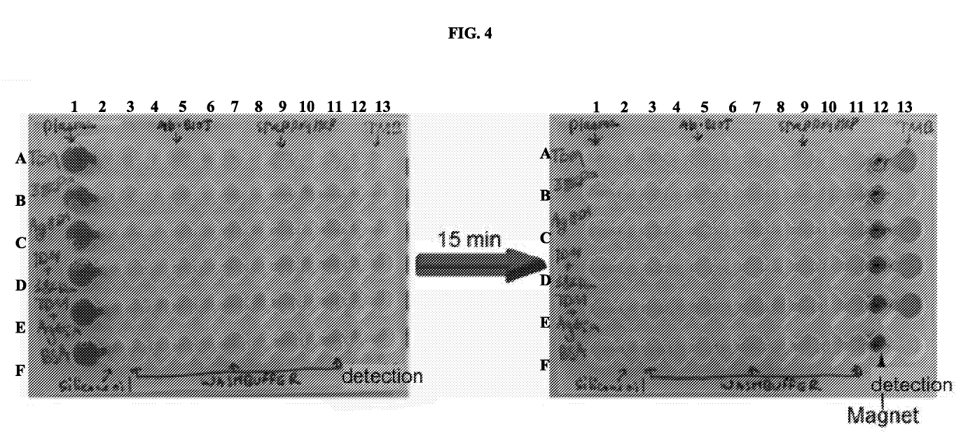

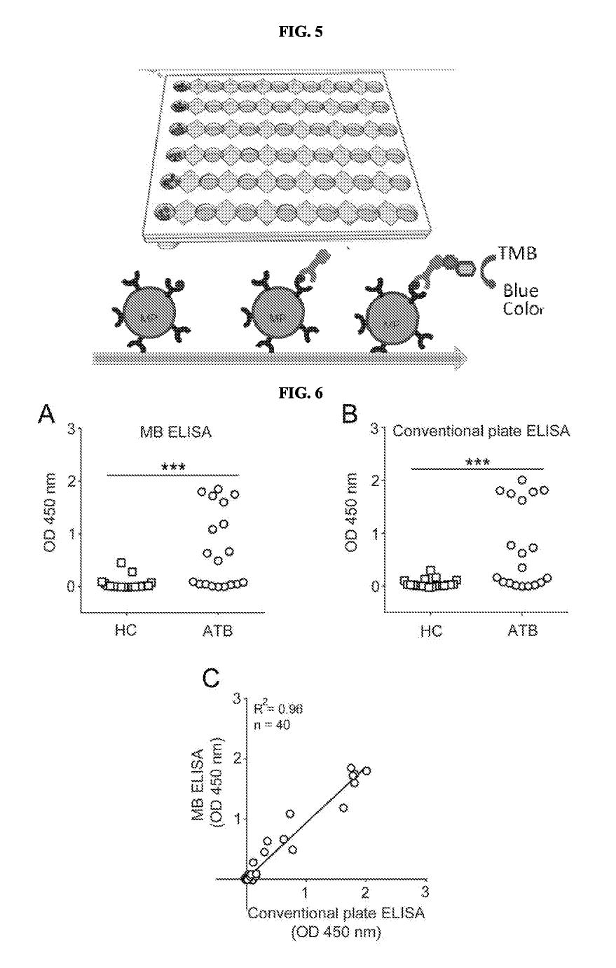

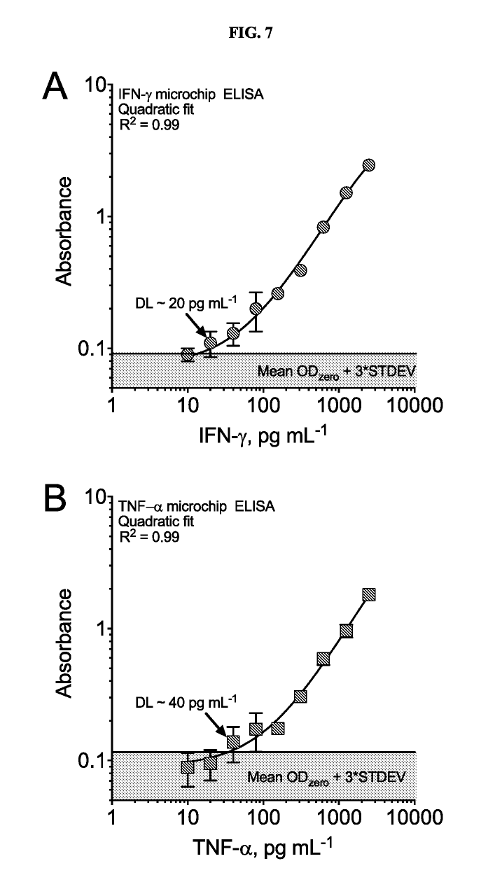

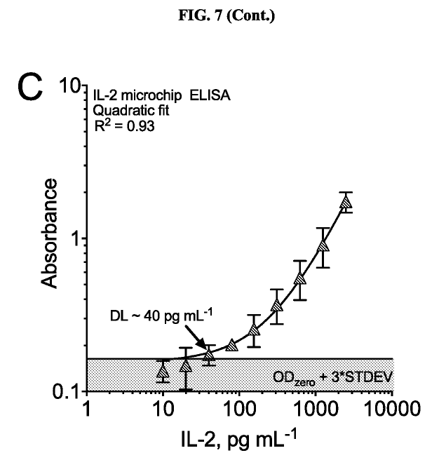

The magnet bead ELISA assay was then translated onto a microchip device for rapid detection of IgG in plasma samples. Each microchip has six channels in which six different reactions/assays can be performed. Each channel consists of connected chambers, which are filled with spatially arranged, stationary, aqueous reagents separated by immiscible oil. The microchip ELISA is carried out by actuating the antigen-coated magnetic beads in each of these chambers using the magnet underneath. Each chamber performs different functions; the first is used for IgG capture, the second for binding of biotin-labelled secondary anti-IgG antibody, and the third for binding of streptavidin polymeric enzyme, with alternate chambers for washing (FIG. 2A). Finally, the MB-bound polymeric enzyme induces TMB oxidation in the latter chamber, generating a blue coloured substrate (FIG. 4). The reaction is stopped and the optical density (OD) is measured at 450 nm. In the first three channels of the microchip ELISA, IgG against TDM, 38 kDa, and Ag85A, respectively, was detected. The fourth and fifth channels were used to measure the total IgG response against TDM combined with each protein antigen. The sixth channel was used as negative control with no antigen added. The entire microchip ELISA process requires 15 min from plasma sample addition to colorimetric detection.

Microchip ELISA Principle.

The microchip device consisted of six channels with each channel featuring seven circular chambers interconnected alternately with six rhombus chambers. The circular chambers contained 70 μl of aqueous reagents, e.g. lipid/protein/antibody-coated magnetic beads (MBs), wash buffer, detection tracer antibody, polymeric enzyme labels and colorimetric substrate, whereas the rhombus chambers contained 70 μl of immiscible silicone oil. The oil provided a barrier between the aqueous reagents, and permits manually assisted magnetic actuation of beads between the reagents. Plasma and reagents were diluted in 5% fatty acid free BSA in PBS buffer.

For simultaneous detection of IgG response against multiple Mycobacterium tuberculosis antigens for tuberculosis diagnosis, in channels 1, 2, and 3 of the microchip device, an equal mixture of TDM-, 38 kDa-, or Ag85A-coated beads (5 μl, 10⁶ beads) and BSA-coated beads (5 μl, 10⁶ beads) were used, whereas channel 4 and 5 contained equal mixtures of TDM-coated MBs (5 μl) and 38 kDa- or Ag85A-coated MBs (5 μl); channel 6 contained BSA-coated MBs (10 μl, 2×10⁶ beads) as a control. Plasma samples (60 μl, 1:200) were then added to the beads and reacted for 3 min.

For development of a cytokine standard curve, 5 independent parafilm bonded microchip devices were used. The experiments were performed at different days and different batches of antibody coated MBs (10 μl, 2×10⁶ beads) were prepared. IFNγ standards (concentration range of 4 IU/ml to 0.25 IU/ml) were prepared by spiking IFNγ in 10% Fetal Bovine Serum. Standards (50 μl, 1:2) were added to the beads and reacted for 10 minutes at 37° C.

For determination of IFN-γ in blood plasma of LTBI and HC individuals, antibody (anti-IFN-γ) coated MBs (10 μl, 2×10⁶ beads) were used in all 6 channels of parafilm bonded microchip. Two wells were allocated for each of the plasma sample from antigen, mitogen and nil tubes. Plasma samples (50 μl, dilution 1:2) were added to the beads and reacted for 10 minutes at 37° C.

For standardization of microchip ELISA to detect cytokines, antibody coated MBs (10 μl, 2×10⁶ beads) were used in all 6 channels of parafilm bonded microchip. Cytokine standards (IFN-γ, TNF-α, or IL-2; at concentration range of 10 to 2500 pg/ml) were spiked in PBS-Tween, RPMI with 10% fetal calf serum (FCS), whole plasma, and whole blood as medium Samples were obtained as pooled plasma from healthy, disease-free individuals. Spiked samples (60 μl) were added to the beads and respectively reacted for 5 minutes, 10 minutes, 10 minutes, and 18 hours at 37° C.

After the capture of specific IgGs or cytokines, beads were magnetically actuated to the wash chamber (30 seconds) to remove nonspecific plasma proteins. Beads were then actuated to the biotin anti-human IgG (500 ng/ml) chamber for biotin-antibody labelling for 2 or 3 minutes. After brief washing (30 seconds), the beads were then actuated to the streptavidin poly-HRP (500 ng/ml) chamber for an additional 2 or 3 min for enzyme labelling. After another wash (30 seconds), beads were actuated to the one-step ultra TMB substrate solution and incubated for 5 minutes. The colorimetric reaction was stopped using an equal volume of 2M H₂SO₄ or 3M H₂SO₄ and the resultant solution was immediately transferred to a 96-well plate for absorbance measurement at 450 nm using a microplate reader (PerkinElmer Envision 2104 multilabel reader). Absorbance values for the test antigen beads (Channel 1-5) were subtracted from the BSA beads (Channel 6), and the difference correlated to the amount of lipid/protein-specific antibodies present in the plasma. The total time of the microchip ELISA assay is about between 15 to 25 minutes.

Data Analysis.

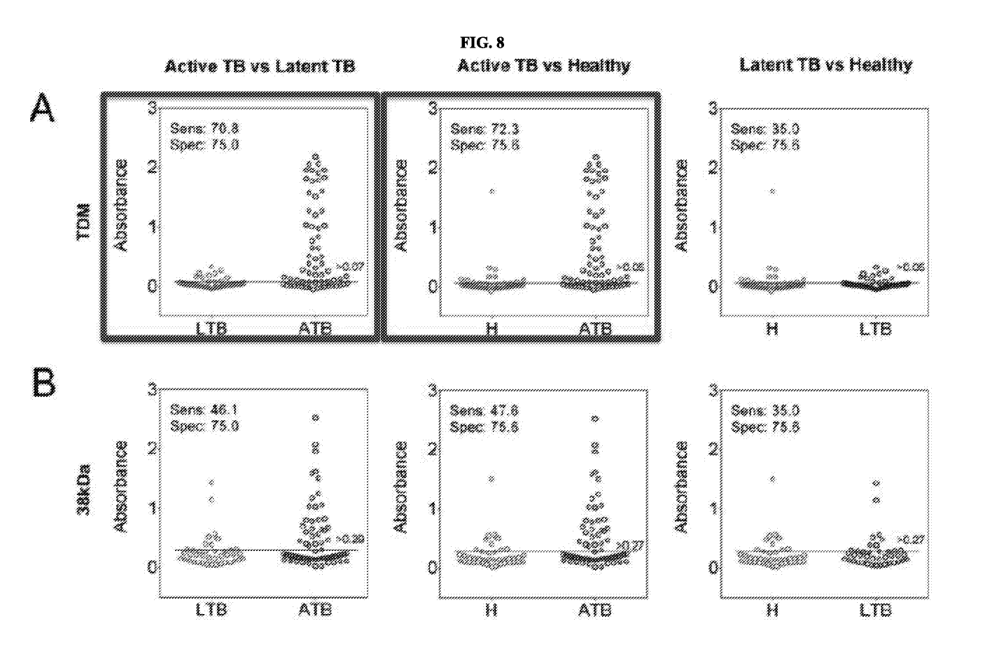

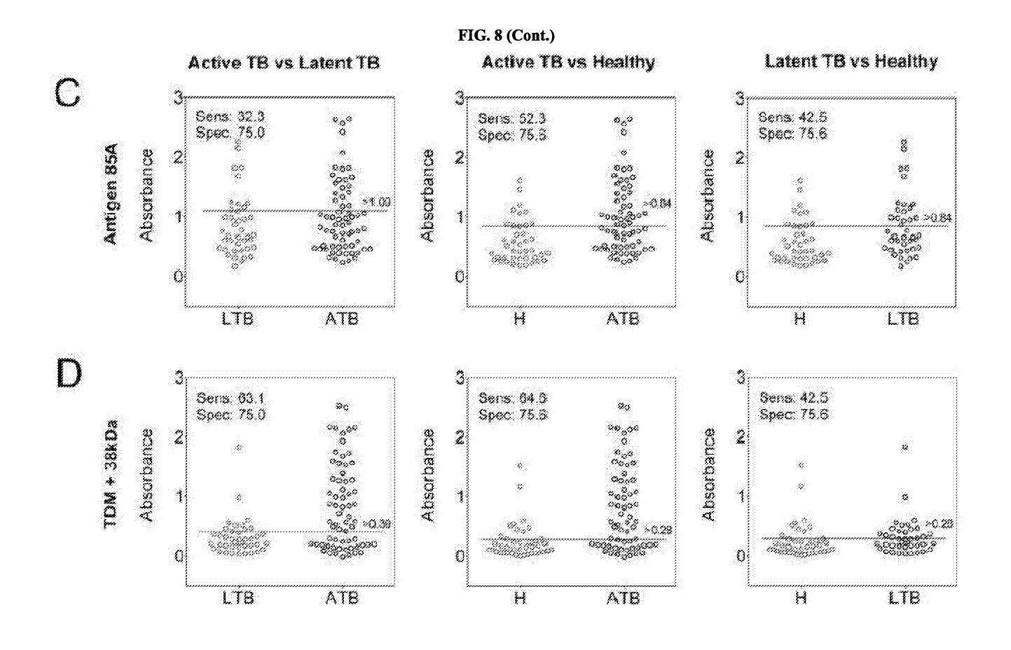

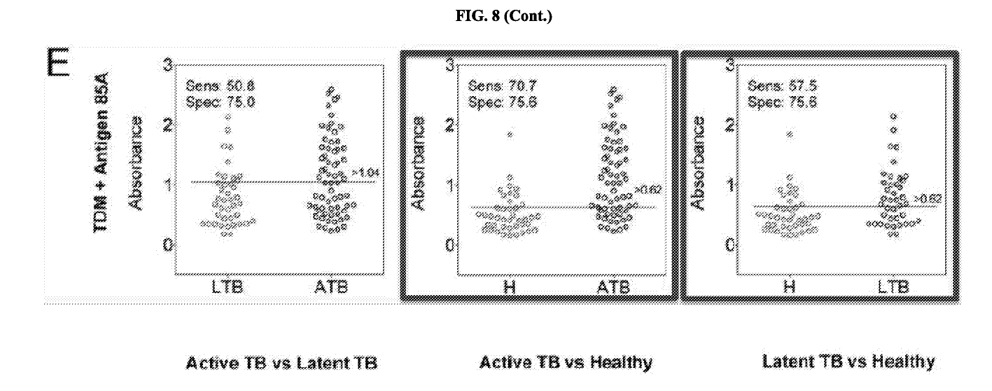

The absorbance values for the microchip and conventional plate or MB-bound assay was measured at 450 nm, by subtracting the values of the control BSA-coated MBs from the test antigen-coated MBs. The cut-off point (horizontal line on the figures, as see in for example FIG. 8) and the sensitivity of the assay were determined by maintaining a constant specificity of 75%. This limit was chosen to compare the sensitivity to multiple antigens at a constant specificity. The ROC curves were generated in GraphPad Prism 5 software, by plotting the true positive rate and the false positive rate. Positive predicting values were obtained from the ratio of true positive samples and the sum of true and false positive samples, whereas negative predicting values were obtained from the ratio of true negative samples and the sum of true and false negative samples. Significant differences in IgG responses between different populations were determined using the Mann-Whitney unpaired t-test (GraphPad Prism 5).

Comparison of MB ELISA with conventional plate ELISA format for anti-TDM response detection. TDM-coated MBs (0.41 μg/cm²) were used to perform a MB ELISA for the detection of anti-TDM IgG in the plasma, and the resultant IgG levels were compared with a conventional plate ELISA format (where TDM is coated on the plate surface). Both formats showed significantly elevated levels of anti-TDM IgG in the plasma of ATB patients compared to HC individuals (FIG. 6 A, B). Specificity of the anti-TDM IgG antibodies was tested using a competitive plate assay where 10% free trehalose was used to block the antigen-antibody reaction. Out of the 22 ATB plasma samples, 12 showed 50% or more inhibition in IgG binding to TDM using free trehalose, suggesting plasma anti-TDM antibody affinity towards the trehalose moiety of TDM (FIG. 17). In the remaining 10 ATB samples, no inhibition by free trehalose was observed, indicating binding of anti-TDM antibodies to epitopes nearby the glycosidic bond between trehalose and mycolic acid made of unique conformations acquired by the disaccharide when bonded to an acyl chain. To benchmark the MB ELISA with a conventional plate ELISA, anti-TDM IgG in the plasma of the same individuals using the two methods was assessed. Both methods showed sensitivity >68% (Table 5) and a good correlation (R²=0.96, FIG. 6C). The overall assay time for the magnetic bead ELISA was about 50 minutes, substantially faster than the conventional plate ELISA (5 h).

### Microchip Based Immunoassay

The microchip-based immunoassay featured the following sequential steps:
Plasma sample was allowed to react with lipid/protein antigen-coated MBs in an aqueous chamber (approx. 3 minutes);
Unbound, non-specific plasma proteins were washed off the beads in the wash chamber (approx. 30 seconds);
Biotin conjugated anti-human IgG was allowed to bind to hIgG adsorbed on the MBs (approx. 2 minutes);
Excess, unbound biotin conjugated anti-human IgG was washed off the beads (approx. 30 seconds);
Streptavidin poly-Horseradish Peroxidase (poly-HRP) was bound to biotin conjugated anti-human IgG on the beads (approx. 2 minutes);
After the immunoreaction, unbound polymeric enzyme was washed of the beads in the wash chamber, and MBs were subsequently transferred to the chamber with TMB substrate; where polymeric enzyme induced oxidation of TMB led to the formation of blue coloured product (approx. 5 minutes);
After colour development the MBs were actuated back to the previous chamber;
Subsequently, stopping solution (75 μl) was added to the TMB chamber and absorbance was measured at 450 nm to determine the levels of IgG antibodies against selected Mtb antigen(s).

The total time of the microchip-based immunoassay was approximately 15 minutes from sample addition to colorimetric detection of antibodies against Mycobacterium tuberculosis.

Antibody Response to Mycobacterium tuberculosis Antigens

The microchip-based immunoassay was used to measure the IgG response against Mycobacterium tuberculosis antigens in the plasma samples of individuals with active tuberculosis (ATB), latent infection (LTB) and healthy control (H) (FIG. 8). A total of 146 plasma samples were tested on the microchip: 65 ATB, 40 LTB and 41 H. IgG response against TDM as biomarker showed the highest sensitivity in discrimination of active tuberculosis samples from those of latent infection as well as healthy control (FIG. 8A). IgG response against mixtures of TDM and Antigen 85A protein showed the highest sensitivity in discrimination of latent individual from healthy control (FIG. 8E).

### ROC Curves for the Antibody Detection Assay

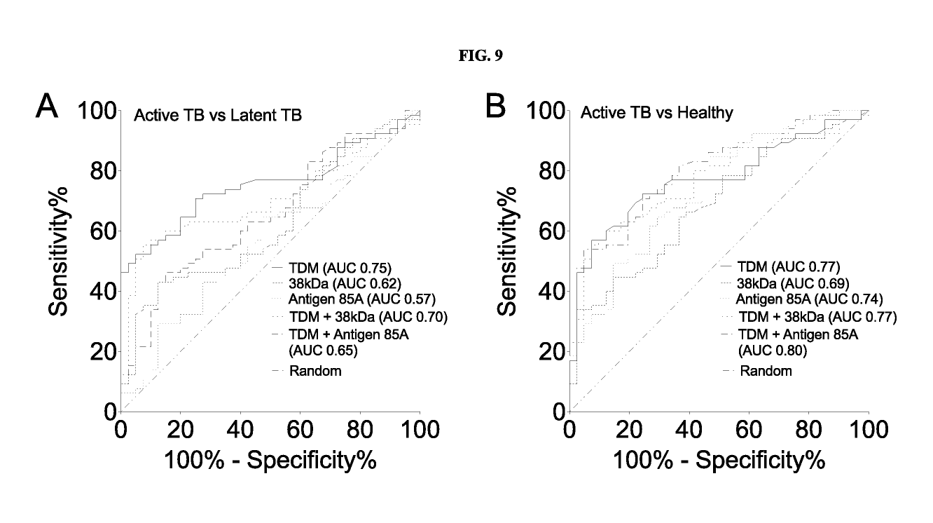

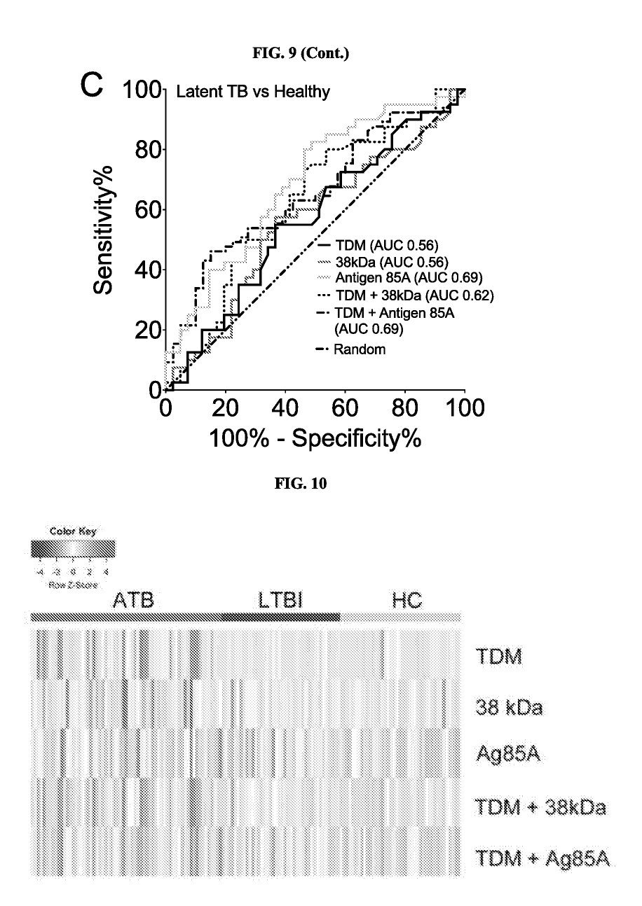

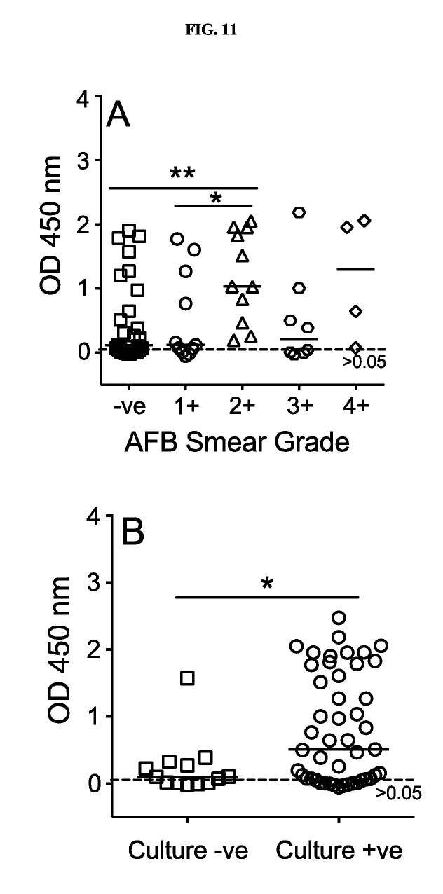

Receiver Operating Characteristic (ROC) curves was generated to illustrate the performance of the three selected antigens to distinguish between active TB, latent infection and healthy control groups. TDM lipid antigen showed the highest area under the curve (AUC) in discrimination of active tuberculosis from latent infection (AUC 0.75; FIG. 9A) and healthy control (AUC 0.77; FIG. 9B). Human IgG responses with TDM as a single antigen was either better or similar to those obtained with combination of two antigens (FIGS. 9A and 9B). Antigen 85A either alone or in combination with TDM lipid showed the highest AUC of 0.69 to discriminate latent infection from healthy controls (FIG. 9C). Thus, Antigen 85A was the strongest predictor in the discrimination of latent from healthy individual.

Comparative Plasma Reactivity to Select Mycobacterium tuberculosis (Mtb) Antigens

To understand the repertoire of responses against the tested antigens, absorbance values were normalized and compared across the ATE, LTB and H individuals. FIG. 10 shows the heat map of the normalized values that indicate plasma reactivity to single and mixture of antigens among the active tuberculosis (ATB), latent tuberculosis (LTB) and healthy (HC) individuals. IgG responses against TDM lipid antigen could discriminate between the active tuberculosis (ATB) cases from those of LTB and H individuals.

### Serodiagnostic Potential of Selected Antigens in Microchip Immunoassay

To decipher the serodiagnostic potential of the tested antigens and their respective combinations in a potential microchip assay based point-of-care (POC) test, the sensitivity, specificity, positive predictive and negative predictive values were calculated. Table 1 indicates that usage of TDM antigen in distinguishing active from latent individuals with a sensitivity and specificity of 71% and 75%, respectively. Likewise, TDM antigen alone was the best performer in distinguishing active TB from healthy individuals, with a sensitivity and specificity of 72% and 76%, respectively (Table 2). Mixtures of TDM lipid and Antigen 85A protein could discriminate, with a sensitivity of 57% and specificity of 76%, between latent tuberculosis infection and healthy individuals (Table 3).

TABLE 1
Evaluation of the serodiagnostic potential of individual TDM lipid, 38 kDa
and Antigen 85A protein, and mixtures of TDM + 38 kDa and TDM + Antigen 85A for
differentiation between ATB and LTB individuals using the microchip ELISA. The generalized
linear model (GLM) was applied to extrapolate the serodiagnostic potential of the combination
of three antigens.
ACTIVE TUBERCULOSIS vs LATENT INFECTION

| | | | POSITIVE | NEGATIVE | |
|---|---|---|---|---|---|
| | | | PREDICTIVE VALUE | PREDICTIVE VALUE | |
| ANTIGEN | SENSITIVITY (%) | SPECIFICITY (%) | (PPV) % | (PPV) % | ROC, AUC |
| TDM | 71 | 75 | 82 | 61 | 0.75 |
| 38 kDa | 46 | 75 | 75 | 46 | 0.62 |
| Antigen 85A | 32 | 75 | 68 | 40 | 0.57 |
| TDM + 38 kDa | 63 | 75 | 81 | 57 | 0.7 |
| TDM + Antigen 85A | 51 | 75 | 77 | 48 | 0.65 |
| GLM of TDM + 38 kDa + Antigen 85A | 65 | 75 | — | — | 0.77 |

TABLE 2
Evaluation of the serodiagnostic potential of TDM, 38 kDa, Antigen 85A,
TDM + 38 kDa, TDM + antigen 85A and combination of three antigens (extrapolated from
GLM) using the microchip ELISA for the differentiation of ATB and H individuals.
ACTIVE TUBERCULOSIS vs LATENT INFECTION

| | | | POSITIVE | NEGATIVE | |
|---|---|---|---|---|---|
| | | | PREDICTIVE VALUE | PREDICTIVE VALUE | |
| ANTIGEN | SENSITIVITY (%) | SPECIFICITY (%) | (PPV) % | (PPV) % | ROC, AUC |
| TDM | 71 | 75 | 82 | 61 | 0.75 |
| 38 kDa | 46 | 75 | 75 | 46 | 0.62 |
| Antigen 85A | 32 | 75 | 68 | 40 | 0.57 |
| TDM + 38 kDa | 63 | 75 | 81 | 57 | 0.7 |
| TDM + Antigen 85A | 51 | 75 | 77 | 48 | 0.65 |
| GLM of TDM + 38 kDa + Antigen 85A | 65 | 75 | — | — | 0.77 |

TABLE 3
Evaluation of serodiagnostic potential of TDM, 38 kDa, Antigen 85A, TDM +
38 kDa, TDM + antigen 85A and combination of three antigens using microchip ELISA for
the differentiation of LTB and H individuals.
LATENT INFECTION vs HEALTHY CONTROL

| | | | POSITIVE | NEGATIVE | |
|---|---|---|---|---|---|
| | | | PREDICTIVE VALUE | PREDICTIVE VALUE | |
| ANTIGEN | SENSITIVITY (%) | SPECIFICITY (%) | (PPV) % | (PPV) % | ROC, AUC |
| TDM | 35 | 76 | 58 | 54 | 0.56 |
| 38 kDa | 35 | 76 | 58 | 54 | 0.56 |
| Antigen 85A | 42 | 76 | 63 | 57 | 0.69 |
| TDM + 38 kDa | 42 | 75 | 63 | 57 | 0.62 |
| TDM + Antigen 85A | 57 | 76 | 70 | 65 | 0.69 |
| GLM of TDM + 38 kDa + Antigen 85A | 56 | 76 | — | — | 0.70 |

Since sputum negative active tuberculosis patients are difficult to diagnose using POC assays, the application of our microchip-based immunoassay to detect this subgroup of active tuberculosis patients was tested. 80%4 of sputum positive and 63% of sputum negative active tuberculosis patients, respectively, were detected on the basis of the anti-TDM IgG response (Table 4).

TABLE 4
Percentage of antigen positive in smear positive and smear
negatives samples.
MINIMUM SPECIFICATIONS FOR A POC FOR
DIAGNOSIS OF TUBERCULOSIS

| | SMEAR-POSITIVE | SMEAR-NEGATIVE |
|---|---|---|
| | CULTURE | CULTURE |
| | CONFIRMED | CONFIRMED |
| ANTIGEN | CASES (%) | CASES (%) |
| TDM | 80 | 63 |
| 38 kDa | 48 | 52 |
| Antigen 85A | 49 | 59 |
| TDM + 38 kDa | 66 | 77 |
| TDM + Antigen 85A | 77 | 67 |
| GLM of TDM + | 77 | 74 |
| 38 kDa + | | |
| Antigen 85A | | |

Next, the serodiagnostic potential of a TDM antigen based microchip ELISA with the minimal specifications specified by World Health Organization (WHO) for a point-of-care test was tested. The comparison indicated that the developed TDM based microchip immunoassay met WHO’ specifications. Given the lower specificity in adults, the developed POCT is intended for triage and referral compared to a test used to make treatment decisions. Furthermore the sensitivity of the immunoassay was better than that of sputum microscopy assay (sensitivity ˜56%) in our cohort.

Simultaneous detection of IgG antibodies against multiple Mtb antigens using microchip ELISA. The microchip ELISA demonstrated detection of IgG antibodies against three antigens (TDM, 38 kDa, and Ag85A) and their combinations in 146 plasma samples (65 ATB, 40 LTBI, and 41 HC). ATB samples were considered positive if the values were higher than the cut-off values obtained from the HC and LTBI samples. The cut-off value for each antigen was set at 75% specificity in order to compare their relative sensitivity to a set specificity. Using the above criteria, 88% (57/65) of the ATB plasma samples were IgG positive against at least one of the three antigens tested. Only 40% (26/65) of the ATB plasma samples were positive for IgG against any two of the antigens tested individually, and only 28% (18/65) of the ATB plasma samples were IgG positive for all three antigens. These findings suggest a heterogeneous IgG response against the three antigens. Compared to HC plasma samples, active tuberculosis (ATB) samples had significantly higher levels of IgG against all three antigens (FIG. 8 A-C), with the highest sensitivity of 72% for TDM, indicating greater reliability of the anti-glycolipid humoral response compared to the anti-protein response in differentiating active tuberculosis (ATB) from healthy control (HC). In addition, receiver operating characteristic (ROC) curve analysis demonstrated a better performance of TDM-based microchip ELISA compared to 38 kDa and Ag85A in discriminating active tuberculosis (ATB) from healthy control (HC) individuals with area under the curve (AUC, 0.77 vs. 0.69 and 0.74, respectively) (FIG. 9B). These results indicate that the IgG humoral immune response to TDM is a promising immunological marker for ATB detection.

Comparison of the TDM based magnetic bead ELISA (MB ELISA) with a conventional plate ELISA assay showed good correlation (R2=0.96; FIG. 6C; Table 5), suggesting that the TDM microchip ELISA is as efficient as a bench top ELISA with the important advantage of being performed in 15 minutes without the requirement for sophisticated instrumentation.

TABE 5
Sensitivity and specificity comparison between conventional plate
ELISA and MB TDM ELISA.

| TDM-coated surface | | |
|---|---|---|
| (time of assay) | Sensitivity (%) | Specificity (%) |
| MB ELISA (~50 min) | 68 | 75 |
| Conventional plate ELISA | 74 | 75 |
| (~5 hr) | | |

A similar trend was observed for differentiating ATB from LTBI individuals. Anti-TDM IgG levels provided the highest discrimination sensitivity (71%) when compared to the responses against the 38 kDa and Ag85A protein antigens (FIG. 8A, left panel and Table 6). Receiver Operating Curves (ROC) further confirmed the superior accuracy of the TDM-based microchip ELISA (AUC, 0.75) compared with the protein antigen-based assay in differentiating active tuberculosis (ATB) from latent tuberculosis individuals (LTBI; Table 6). Indeed, the IgG response against TDM was the strongest predictor for discriminating active tuberculosis from healthy control (HC) and latent tuberculosis individuals (LTBI) according to a generalised linear model (using logistic regression analysis).

TABLE 6
Evaluation of serodiagnostic potential of individual antigens
and their combination using microchip ELISA for the
differentiation of ATB and LTBI individuals.
ATB vs. LTBI

| | | | Positive | Negative | |
|---|---|---|---|---|---|
| | | | predictive | predictive | |
| | Sensitivity | Specificity | value | value | ROC, |
| Antigen | (%) | (%) | (PPV) % | (NPV) % | AUC |
| TDM | 71 | 75 | 82 | 61 | 0.75 |
| 38 kDa | 46 | 75 | 75 | 46 | 0.62 |
| Ag85A | 32 | 75 | 68 | 40 | 0.57 |
| TDM + 38 kDa | 63 | 75 | 81 | 57 | 0.7 |
| TDM + Ag85A | 51 | 75 | 77 | 48 | 0.65 |

1-27. (canceled)

28. An enzyme-linked immunosorbent assay (ELISA) plate comprising at least one row of reaction chambers, wherein the reaction chambers in the same row are in fluid communication with each other, wherein the reaction chambers in the same row comprise a first geometry and a second geometry different from the first geometry; wherein the ELISA plate comprises a base plate and a top plate, wherein the base plate comprises a solid plate and wherein the top plate comprises perforations forming the reaction chambers.

29. The ELISA plate of claim 28, wherein a geometry of the reaction chambers is selected from the group consisting of cuboid, cube, cylindrical, circular, spherical, rectangular, square, triangular, polygonal, rhombic, hexagonal prism, elliptical, ellipsoid or trapezoidal.

30. The ELISA plate of claim 28, wherein the first geometry is circular and the second geometry is selected from the group consisting of rhombic, square, circular and ellipsoid, optionally wherein the ELISA plate further comprising a hydrophobic layer disposed between the base plate and the top plate.

31. A method of performing an enzyme-linked immunosorbent assay (ELISA) using a system for detecting a target analyte comprising an ELISA plate comprising at least one row of reaction chambers, wherein the reaction chambers in the same row are in fluid communication with each other, wherein the reaction chambers in the same row comprise a first geometry and a second geometry different from the first geometry; wherein the ELISA plate comprises a base plate and a top plate, wherein the base plate comprises a solid plate and wherein the top plate comprises perforations forming the reaction chambers,
a plurality of magnetic beads, and
a magnet configured to cooperate with the magnetic beads,
wherein the reaction chambers of the ELISA plate are liquid-filled, the method comprising:
(a) incubating a sample comprising one or more target analytes with a plurality of magnetic beads capable of capturing said one or more target analytes in the first chamber of each row of the ELISA plate according to any of the preceding claims;
(b) loading the subsequent reaction chambers of the columns of the ELISA plate with alternating liquids, wherein the liquids are either aqueous or non-aqueous;
(c) moving the plurality of magnetic beads from the first reaction chambers of each row to subsequent reaction chambers of the same row by using the magnet;
(d) incubating the plurality of magnetic beads in subsequent reaction chambers;
(e) repeating operations (c) to (d) until the final chamber in the row is reached; and
(f) detecting the signal generated in the final reaction chamber.

32. The method of claim 31, wherein one or more intermediate measurements are made between the first reaction chamber and the final reaction chamber of each row.

33. The method of claim 31, wherein the reaction chambers are filled with alternating liquids.

34. The method of claim 31, wherein the reaction chambers are filled with alternating liquids, wherein the alternating liquids are non-aqueous and aqueous.

35. The method of claim 31, wherein the reaction chambers are filled with alternating liquids, wherein the alternating liquids are non-aqueous and aqueous, and wherein the non-aqueous liquid is a non-polar liquid.

36. The method of claim 31, wherein the reaction chambers are filled with alternating liquids, wherein the alternating liquids are non-aqueous and aqueous, and wherein the non-aqueous liquid is non-polar liquid selected from the group consisting of mineral oil, silicone oil, linseed oil, sunflower oil, rapeseed oil and paraffin.

37. The method of claim 31, wherein the reaction chambers are filled with alternating liquids, wherein the alternating liquids are non-aqueous and aqueous, and wherein the aqueous liquid is selected from the group consisting of a wash buffer, a primary antibody solution, a secondary antibody solution, an enzyme solution and a colorimetric detection solution.

38. A method of detecting tuberculosis in a subject or at least one cytokine in a sample using a system for detecting a target analyte comprising an ELISA plate comprising at least one row of reaction chambers, wherein the reaction chambers in the same row are in fluid communication with each other, wherein the reaction chambers in the same row comprise a first geometry and a second geometry different from the first geometry; wherein the ELISA plate comprises a base plate and a top plate, wherein the base plate comprises a solid plate and wherein the top plate comprises perforations forming the reaction chambers,
a plurality of magnetic beads, and
a magnet configured to cooperate with the magnetic beads.

39. The method of claim 38, wherein the at least one cytokine is selected from the group consisting of chemokines, interferons, interleukins, lymphokines and tumour necrosis factors.

40. The method of claim 38, wherein the at least one cytokine is interferon, wherein the interferon is IFN-γ.

41. The method of claim 38, wherein the at least one cytokine is tumour necrosis factor, wherein the tumour necrosis factor is TNF-α.

42. The method of claim 38, wherein the at least one cytokine is interleukin, wherein the interleukin is IL-2.

43. The method of claim 38, wherein the sample is a sample obtained from a subject or from cell culture.
